(19) United States
(12) Patent Application Publication
(10) Pub. No.: US 2026/0364119 A1
(43) Pub. Date: Nov. 19, 2026

(54) RETINAL PIGMENT EPITHELIUM CELL THERAPY

(71) Applicant: The Regents of the University of California, Oakland, CA (US)

(72) Inventor: Steven Daniel Schwartz, Los Angeles, CA (US)

(73) Assignee: The Regents of the University of California, Oakland, CA (US)

(21) Appl. No.: 19/633,521

(22) Filed: Mar. 30, 2026

Related U.S. Application Data
(63) Continuation of application No. 19/478,747, filed on May 3, 2024.
(60) Provisional application No. 63/500,482, filed on May 5, 2023.

(51) Int. Cl. A61K 35/30; A61K 9/00; A61P 27/02; C12N 5/079

(52) U.S. Cl. CPC A61K 35/30; A61K 9/0048; A61P 27/02; C12N 5/0621; C12N 2506/1307

(57) ABSTRACT

Compositions of retinal pigment epithelium (RPE) cells, such as autologous induced pluripotent stem cell-derived RPE cells, and their use in treating eye disorders are described.

### CROSS-REFERENCE TO RELATED APPLICATIONS

This application is a continuation application which claims the benefit under 35 U.S.C. § 120 and § 121 of U.S. patent application Ser. No. 19/478,747, filed Oct. 24, 2025, which is a U.S. national phase of International Application Serial No. PCT/US24/27790, filed on May 3, 2024, which application claims the benefit under 35 U.S.C. Section 119 (e) of U.S. Provisional Patent Application Ser. No. 63/500,482, filed on May 5, 2023, all of which are incorporated in their entirety by reference herein.

### BACKGROUND

Several degenerative diseases of the eye cause permanent vision loss due to retinal pigment epithelial (RPE) cell dysfunction. Treatments for these diseases represent a major unmet medical need.

### SUMMARY

The present disclosure provides, among other things, a preparation comprising retinal pigment epithelium (RPE) cells differentiated from induced pluripotent stem cells (iPSCs), wherein at least 80% (e.g., at least 85%, 90%, 91%, 92%, 93%, 94%, 95% or more) of the RPE cells are PMEL17 (+)/CD140b (+)/GD2 (−)/CD184 (−). In some embodiments, about 80% (e.g., about 85%, 90%, 91%, 92%, 93%, 94%, 95% or more) of the RPE cells are PMEL17 (+)/CD140b (+)/GD2 (−)/CD184 (−).

In some embodiments, at least 80% (e.g., at least 85%, 90%, 91%, 92%, 93%, 94%, 95% or more) of the RPE cells express RPE65, BEST1, RLBP1, and MerTK. In some embodiments, about 80% (e.g., about 85%, 90%, 91%, 92%, 93%, 94%, 95% or more) of the RPE cells express RPE65, BEST1, RLBP1, and MerTK.

In some embodiments, fewer than about 0.002% of the RPE cells are positive for Lin28, Oct4, or Klf4, e.g., as detected by digital droplet PCR (ddPCR). In some embodiments, fewer than about 0.002% of the RPE cells are positive for Lin28, and the RPE cells do not have measurable expression of Oct4, or Klf4, as detected by digital droplet PCR (ddPCR).

In some embodiments, a culture of the preparation comprises at least 95% (e.g., at least 96%, 97%, 98%, 99% or more) of RPE cells exhibiting confluent hexagonal cells, pigmentation, cobblestone configuration, well-defined membrane borders, and/or polarization (e.g., as assessed using Ezrin staining). In some embodiments, a culture of the preparation comprises at least 95% (e.g., at least 96%, 97%, 98%, 99% or more) of RPE cells exhibiting confluent hexagonal cells, pigmentation, cobblestone configuration, and well-defined membrane borders. In some embodiments, a culture of the preparation comprises at least 95% (e.g., at least 96%, 97%, 98%, 99% or more) of RPE cells exhibiting confluent hexagonal cells, pigmentation, cobblestone configuration, well-defined membrane borders, and polarization (e.g., as assessed using Ezrin staining). In some embodiments, a culture of the preparation comprises about 95% (e.g., about 96%, 97%, 98%, 99% or more) of RPE cells exhibiting confluent hexagonal cells, pigmentation, cobblestone configuration, well-defined membrane borders, and/or polarization (e.g., as assessed using Ezrin staining). In some embodiments, a culture of the preparation comprises about 95% (e.g., about 96%, 97%, 98%, 99% or more) of RPE cells exhibiting confluent hexagonal cells, pigmentation, cobblestone configuration, and well-defined membrane borders. In some embodiments, a culture of the preparation comprises about 95% (e.g., about 96%, 97%, 98%, 99% or more) of RPE cells exhibiting confluent hexagonal cells, pigmentation, cobblestone configuration, well-defined membrane borders, and polarization (e.g., as assessed using Ezrin staining).

In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and/or (d) at least 50% of the RPE cells exhibit ability to phagocytose photoreceptor outer segments, e.g., as measured by flow cytometry. In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and (d) at least 50% of the RPE cells exhibit ability to phagocytose photoreceptor outer segments, e.g., as measured by flow cytometry.

In some embodiments, upon passage through a transplantation device (e.g., a 41 g device), at least 80% (e.g., at least 85%, 90%, 91%, 92%, 93%, 94%, 95% or more) of the RPE cells are viable, e.g., as determined by trypan blue staining. In some embodiments, upon passage through a transplantation device (e.g., a 41 g device), about 80% (e.g., about 85%, 90%, 91%, 92%, 93%, 94%, 95% or more) of the RPE cells are viable, e.g., as determined by trypan blue staining.

In some embodiments, upon passage through a transplantation device (e.g., a 41 g device), at least 80% (e.g., at least 85%, 90%, 91%, 92%, 93%, 94%, 95% or more) of the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture. In some embodiments, upon passage through a transplantation device (e.g., a 41 g device), about 80% (e.g., about 85%, 90%, 91%, 92%, 93%, 94%, 95% or more) of the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture.

In some embodiments, upon storage at room temperature for about 6 hours, at least 80% (e.g., at least 85%, 90%, 91%, 92%, 93%, 94%, 95% or more) of the RPE cells are viable, e.g., as determined by trypan blue staining. In some embodiments, upon storage at room temperature for about 6 hours, about 80% (e.g., about 85%, 90%, 91%, 92%, 93%, 94%, 95% or more) of the RPE cells are viable, e.g., as determined by trypan blue staining.

In some embodiments, the iPSCs are derived from fibroblasts or from peripheral blood mononuclear cells.

In some embodiments, the preparation comprises a suspension of the RPE cells. In some embodiments, the preparation comprises a scaffold, matrix, or bio ink, wherein the RPE cells are seeded onto, cultured onto, printed onto, or embedded in, the scaffold, matrix, or bio ink.

In some embodiments, the preparation comprises about 10,000 to about 1,500,000 RPE cells.

In another aspect, the disclosure provides a preparation comprising retinal pigment epithelium (RPE) cells differentiated from induced pluripotent stem cells (iPSCs), wherein the preparation has undergone at least one freeze/thaw cycle and has at least 80%, 90%, or 95% viability (e.g., as determined by trypan blue staining), relative to a reference preparation comprising RPE cells differentiated from iPSCs and not having undergone at least one freeze/thaw cycle. In some embodiments, the preparation has undergone at least one freeze/thaw cycle and has about 80%, 90%, or 95% viability (e.g., as determined by trypan blue staining), relative to a reference preparation comprising RPE cells differentiated from iPSCs and not having undergone at least one freeze/thaw cycle. In some embodiments, following at least one freeze/thaw cycle of the preparation, at least 80%, 90%, or 95% of the RPE cells of the preparation are viable (e.g., as determined by trypan blue staining). In some embodiments, following at least one freeze/thaw cycle of the preparation, about 80%, 90%, or 95% of the RPE cells of the preparation are viable (e.g., as determined by trypan blue staining). In some embodiments, following at least one freeze/thaw cycle, the preparation has undergone culture in culture media for about 2 weeks to about 6 weeks, e.g., for about 4 weeks.

In some embodiments, at least 80% (e.g., at least 85%, 90%, 91%, 92%, 93%, 94%, 95% or more) of the RPE cells are PMEL17 (+)/CD140b (+)/GD2 (−)/CD184 (−). In some embodiments, about 80% (e.g., about 85%, 90%, 91%, 92%, 93%, 94%, 95% or more) of the RPE cells are PMEL17 (+)/CD140b (+)/GD2 (−)/CD184 (−).

In some embodiments, at least 80% (e.g., at least 85%, 90%, 91%, 92%, 93%, 94%, 95% or more) of the RPE cells express RPE65, BEST1, RLBP1, and MerTK. In some embodiments, about 80% (e.g., about 85%, 90%, 91%, 92%, 93%, 94%, 95% or more) of the RPE cells express RPE65, BEST1, RLBP1, and MerTK.

In some embodiments, fewer than about 0.002% of the RPE cells are positive for Lin28, Oct4, or Klf4, e.g., as detected by digital droplet PCR (ddPCR). In some embodiments, fewer than about 0.002% of the RPE cells are positive for Lin28, and the RPE cells do not have measurable expression of Oct4, or Klf4, as detected by digital droplet PCR (ddPCR).

In some embodiments, a culture of the preparation comprises at least 95% (e.g., at least 96%, 97%, 98%, 99% or more) of RPE cells exhibiting confluent hexagonal cells, pigmentation, cobblestone configuration, well-defined membrane borders, and/or polarization (e.g., as assessed using Ezrin staining). In some embodiments, a culture of the preparation comprises at least 95% (e.g., at least 96%, 97%, 98%, 99% or more) of RPE cells exhibiting confluent hexagonal cells, pigmentation, cobblestone configuration, and well-defined membrane borders. In some embodiments, a culture of the preparation comprises at least 95% (e.g., at least 96%, 97%, 98%, 99% or more) of RPE cells exhibiting confluent hexagonal cells, pigmentation, cobblestone configuration, well-defined membrane borders, and polarization (e.g., as assessed using Ezrin staining). In some embodiments, a culture of the preparation comprises about 95% (e.g., about 96%, 97%, 98%, 99% or more) of RPE cells exhibiting confluent hexagonal cells, pigmentation, cobblestone configuration, well-defined membrane borders, and/or polarization (e.g., as assessed using Ezrin staining). In some embodiments, a culture of the preparation comprises about 95% (e.g., about 96%, 97%, 98%, 99% or more) of RPE cells exhibiting confluent hexagonal cells, pigmentation, cobblestone configuration, and well-defined membrane borders. In some embodiments, a culture of the preparation comprises about 95% (e.g., about 96%, 97%, 98%, 99% or more) of RPE cells exhibiting confluent hexagonal cells, pigmentation, cobblestone configuration, well-defined membrane borders, and polarization (e.g., as assessed using Ezrin staining).

In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and/or (d) at least 50% of the RPE cells exhibit ability to phagocytose photoreceptor outer segments, e.g., as measured by flow cytometry. In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and (d) at least 50% of the RPE cells exhibit ability to phagocytose photoreceptor outer segments, e.g., as measured by flow cytometry.

In some embodiments, upon passage through a transplantation device (e.g., a 41 g device), at least 80% (e.g., at least 85%, 90%, 91%, 92%, 93%, 94%, 95% or more) of the RPE cells are viable, e.g., as determined by trypan blue staining. In some embodiments, upon passage through a transplantation device (e.g., a 41 g device), about 80% (e.g., about 85%, 90%, 91%, 92%, 93%, 94%, 95% or more) of the RPE cells are viable, e.g., as determined by trypan blue staining.

In some embodiments, upon passage through a transplantation device (e.g., a 41 g device), at least 80% (e.g., at least 85%, 90%, 91%, 92%, 93%, 94%, 95% or more) of the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture. In some embodiments, upon passage through a transplantation device (e.g., a 41 g device), about 80% (e.g., about 85%, 90%, 91%, 92%, 93%, 94%, 95% or more) of the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture.

In some embodiments, upon storage at room temperature for about 6 hours, at least 80% (e.g., at least 85%, 90%, 91%, 92%, 93%, 94%, 95% or more) of the RPE cells are viable, e.g., as determined by trypan blue staining. In some embodiments, upon storage at room temperature for about 6 hours, about 80% (e.g., about 85%, 90%, 91%, 92%, 93%, 94%, 95% or more) of the RPE cells are viable, e.g., as determined by trypan blue staining.

In some embodiments, the iPSCs are derived from fibroblasts or from peripheral blood mononuclear cells.

In some embodiments, the preparation comprises a suspension of the RPE cells. In some embodiments, the preparation comprises a scaffold, matrix, or bio ink, wherein the RPE cells are seeded onto, cultured onto, printed onto, or embedded in, the scaffold, matrix, or bio ink.

In some embodiments, the preparation comprises about 10,000 to about 1,500,000 RPE cells.

In another aspect, the disclosure provides a method of manufacturing a preparation of RPE cells, the method comprising: propagating fibroblasts from a skin sample or propagating peripheral blood mononuclear cells from a whole blood sample; producing iPSCs from the fibroblasts or from the peripheral blood mononuclear cells; differentiating the iPSCs into a culture of RPE cells; and passaging the culture of RPE cells for at least 10 minutes using a dissociation reagent until at least 95% (e.g., about 95%, about 96%, about 97%, about 98%, about 99% or more) of the cultured RPE cells exhibit confluent hexagonal cells, pigmentation, cobblestone configuration, and well defined membrane borders, thereby producing a preparation of RPE cells. In some embodiments, the method further comprising subjecting the preparation to at least one freeze/thaw cycle.

In some embodiments, at least 80% (e.g., at least 85%, 90%, 91%, 92%, 93%, 94%, 95% or more) of the RPE cells of the preparation are PMEL17 (+)/CD140b (+)/GD2 (−)/CD184 (−). In some embodiments, about 80% (e.g., about 85%, 90%, 91%, 92%, 93%, 94%, 95% or more) of the RPE cells of the preparation are PMEL17 (+)/CD140b (+)/GD2 (−)/CD184 (−).

In some embodiments, at least 80% (e.g., at least 85%, 90%, 91%, 92%, 93%, 94%, 95% or more) of the RPE cells of the preparation express RPE65, BEST1, RLBP1, and MerTK. In some embodiments, about 80% (e.g., about 85%, 90%, 91%, 92%, 93%, 94%, 95% or more) of the RPE cells of the preparation express RPE65, BEST1, RLBP1, and MerTK.

In some embodiments, fewer than about 0.002% of the RPE cells of the preparation are positive for Lin28, Oct4, or Klf4, e.g., as detected by digital droplet PCR (ddPCR). In some embodiments, fewer than about 0.002% of the RPE cells of the preparation are positive for Lin28, and the RPE cells do not have measurable expression of Oct4, or Klf4, as detected by digital droplet PCR (ddPCR).

In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and/or (d) at least 50% of the RPE cells exhibit ability to phagocytose photoreceptor outer segments, e.g., as measured by flow cytometry. In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and (d) at least 50% of the RPE cells exhibit ability to phagocytose photoreceptor outer segments, e.g., as measured by flow cytometry.

In some embodiments, upon passage through a transplantation device (e.g., a 41 g device), at least 80% (e.g., at least 85%, 90%, 91%, 92%, 93%, 94%, 95% or more) of the RPE cells are viable, e.g., as determined by trypan blue staining. In some embodiments, upon passage through a transplantation device (e.g., a 41 g device), about 80% (e.g., about 85%, 90%, 91%, 92%, 93%, 94%, 95% or more) of the RPE cells are viable, e.g., as determined by trypan blue staining.

In some embodiments, upon storage at room temperature for about 6 hours, at least 80% (e.g., at least 85%, 90%, 91%, 92%, 93%, 94%, 95% or more) of the RPE cells are viable, e.g., as determined by trypan blue staining. In some embodiments, upon storage at room temperature for about 6 hours, about 80% (e.g., about 85%, 90%, 91%, 92%, 93%, 94%, 95% or more) of the RPE cells are viable, e.g., as determined by trypan blue staining.

In another aspect, the disclosure provides a preparation of RPE cells produced by any of the methods described herein.

In another aspect, the disclosure provides a method of treating a subject suffering from or at risk of an eye disorder, the method comprising administering to an eye of the subject a preparation of RPE cells. In some embodiments, the RPE cells are autologous to the subject. In some embodiments, the preparation is introduced into any region of the eye of the subject.

In some embodiments, the preparation comprises RPE cells differentiated from induced pluripotent stem cells (iPSCs), wherein at least 80% (e.g., at least 85%, 90%, 91%, 92%, 93%, 94%, 95% or more) of the RPE cells are PMEL17 (+)/CD140b (+)/GD2 (−)/CD184 (−). In some embodiments, about 80% (e.g., about 85%, 90%, 91%, 92%, 93%, 94%, 95% or more) of the RPE cells are PMEL17 (+)/CD140b (+)/GD2 (−)/CD184 (−).

In some embodiments, at least 80% (e.g., at least 85%, 90%, 91%, 92%, 93%, 94%, 95% or more) of the RPE cells express RPE65, BEST1, RLBP1, and MerTK. In some embodiments, about 80% (e.g., about 85%, 90%, 91%, 92%, 93%, 94%, 95% or more) of the RPE cells express RPE65, BEST1, RLBP1, and MerTK.

In some embodiments, fewer than about 0.002% of the RPE cells are positive for Lin28, Oct4, or Klf4, e.g., as detected by digital droplet PCR (ddPCR). In some embodiments, fewer than about 0.002% of the RPE cells are positive for Lin28, and the RPE cells do not have measurable expression of Oct4, or Klf4, as detected by digital droplet PCR (ddPCR).

In some embodiments, a culture of the preparation comprises at least 95% (e.g., at least 96%, 97%, 98%, 99% or more) of RPE cells exhibiting confluent hexagonal cells, pigmentation, cobblestone configuration, well-defined membrane borders, and/or polarization (e.g., as assessed using Ezrin staining). In some embodiments, a culture of the preparation comprises at least 95% (e.g., at least 96%, 97%, 98%, 99% or more) of RPE cells exhibiting confluent hexagonal cells, pigmentation, cobblestone configuration, and well-defined membrane borders. In some embodiments, a culture of the preparation comprises at least 95% (e.g., at least 96%, 97%, 98%, 99% or more) of RPE cells exhibiting confluent hexagonal cells, pigmentation, cobblestone configuration, well-defined membrane borders, and polarization (e.g., as assessed using Ezrin staining). In some embodiments, a culture of the preparation comprises about 95% (e.g., about 96%, 97%, 98%, 99% or more) of RPE cells exhibiting confluent hexagonal cells, pigmentation, cobblestone configuration, well-defined membrane borders, and/or polarization (e.g., as assessed using Ezrin staining). In some embodiments, a culture of the preparation comprises about 95% (e.g., about 96%, 97%, 98%, 99% or more) of RPE cells exhibiting confluent hexagonal cells, pigmentation, cobblestone configuration, and well-defined membrane borders. In some embodiments, a culture of the preparation comprises about 95% (e.g., about 96%, 97%, 98%, 99% or more) of RPE cells exhibiting confluent hexagonal cells, pigmentation, cobblestone configuration, well-defined membrane borders, and polarization (e.g., as assessed using Ezrin staining).

In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and/or (d) at least 50% of the RPE cells exhibit ability to phagocytose photoreceptor outer segments, e.g., as measured by flow cytometry. In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and (d) at least 50% of the RPE cells exhibit ability to phagocytose photoreceptor outer segments, e.g., as measured by flow cytometry.

In some embodiments, upon passage through a transplantation device (e.g., a 41 g device), at least 80% (e.g., at least 85%, 90%, 91%, 92%, 93%, 94%, 95% or more) of the RPE cells are viable, e.g., as determined by trypan blue staining. In some embodiments, upon passage through a transplantation device (e.g., a 41 g device), about 80% (e.g., about 85%, 90%, 91%, 92%, 93%, 94%, 95% or more) of the RPE cells are viable, e.g., as determined by trypan blue staining.

In some embodiments, upon passage through a transplantation device (e.g., a 41 g device), at least 80% (e.g., at least 85%, 90%, 91%, 92%, 93%, 94%, 95% or more) of the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture. In some embodiments, upon passage through a transplantation device (e.g., a 41 g device), about 80% (e.g., about 85%, 90%, 91%, 92%, 93%, 94%, 95% or more) of the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture.

In some embodiments, upon storage at room temperature for about 6 hours, at least 80% (e.g., at least 85%, 90%, 91%, 92%, 93%, 94%, 95% or more) of the RPE cells are viable, e.g., as determined by trypan blue staining. In some embodiments, upon storage at room temperature for about 6 hours, about 80% (e.g., about 85%, 90%, 91%, 92%, 93%, 94%, 95% or more) of the RPE cells are viable, e.g., as determined by trypan blue staining.

In some embodiments, the iPSCs are derived from fibroblasts or from peripheral blood mononuclear cells.

In some embodiments, the preparation comprises a suspension of the RPE cells. In some embodiments, the preparation comprises a scaffold, matrix, or bio ink, wherein the RPE cells are seeded onto, cultured onto, printed onto, or embedded in, the scaffold, matrix, or bio ink.

In some embodiments, the preparation comprises about 10,000 to about 1,500,000 RPE cells.

In another aspect, the disclosure provides a method of treating a subject suffering from or at risk of an eye disorder, the method comprising administering to an eye of the subject a preparation of RPE cells. In some embodiments, the RPE cells are autologous to the subject. In some embodiments, the preparation is introduced into any region of the eye of the subject.

In some embodiments, the preparation comprises RPE cells differentiated from induced pluripotent stem cells (iPSCs), wherein the preparation has undergone at least one freeze/thaw cycle and has at least 80%, 90%, or 95% viability (e.g., as determined by trypan blue staining), relative to a reference preparation comprising RPE cells differentiated from iPSCs and not having undergone at least one freeze/thaw cycle. In some embodiments, the preparation has undergone at least one freeze/thaw cycle and has about 80%, 90%, or 95% viability (e.g., as determined by trypan blue staining), relative to a reference preparation comprising RPE cells differentiated from iPSCs and not having undergone at least one freeze/thaw cycle. In some embodiments, following at least one freeze/thaw cycle of the preparation, at least 80%, 90%, or 95% of the RPE cells of the preparation are viable (e.g., as determined by trypan blue staining). In some embodiments, following at least one freeze/thaw cycle of the preparation, about 80%, 90%, or 95% of the RPE cells of the preparation are viable (e.g., as determined by trypan blue staining). In some embodiments, following at least one freeze/thaw cycle, the preparation has undergone culture in culture media for about 2 weeks to about 6 weeks, e.g., for about 4 weeks.

In some embodiments, at least 80% (e.g., at least 85%, 90%, 91%, 92%, 93%, 94%, 95% or more) of the RPE cells are PMEL17 (+)/CD140b (+)/GD2 (−)/CD184 (−). In some embodiments, about 80% (e.g., about 85%, 90%, 91%, 92%, 93%, 94%, 95% or more) of the RPE cells are PMEL17 (+)/CD140b (+)/GD2 (−)/CD184 (−).

In some embodiments, at least 80% (e.g., at least 85%, 90%, 91%, 92%, 93%, 94%, 95% or more) of the RPE cells express RPE65, BEST1, RLBP1, and MerTK. In some embodiments, about 80% (e.g., about 85%, 90%, 91%, 92%, 93%, 94%, 95% or more) of the RPE cells express RPE65, BEST1, RLBP1, and MerTK.

In some embodiments, fewer than about 0.002% of the RPE cells are positive for Lin28, Oct4, or Klf4, e.g., as detected by digital droplet PCR (ddPCR). In some embodiments, fewer than about 0.002% of the RPE cells are positive for Lin28, and the RPE cells do not have measurable expression of Oct4, or Klf4, as detected by digital droplet PCR (ddPCR).

In some embodiments, a culture of the preparation comprises at least 95% (e.g., at least 96%, 97%, 98%, 99% or more) of RPE cells exhibiting confluent hexagonal cells, pigmentation, cobblestone configuration, well-defined membrane borders, and/or polarization (e.g., as assessed using Ezrin staining). In some embodiments, a culture of the preparation comprises at least 95% (e.g., at least 96%, 97%, 98%, 99% or more) of RPE cells exhibiting confluent hexagonal cells, pigmentation, cobblestone configuration, and well-defined membrane borders. In some embodiments, a culture of the preparation comprises at least 95% (e.g., at least 96%, 97%, 98%, 99% or more) of RPE cells exhibiting confluent hexagonal cells, pigmentation, cobblestone configuration, well-defined membrane borders, and polarization (e.g., as assessed using Ezrin staining). In some embodiments, a culture of the preparation comprises about 95% (e.g., about 96%, 97%, 98%, 99% or more) of RPE cells exhibiting confluent hexagonal cells, pigmentation, cobblestone configuration, well-defined membrane borders, and/or polarization (e.g., as assessed using Ezrin staining). In some embodiments, a culture of the preparation comprises about 95% (e.g., about 96%, 97%, 98%, 99% or more) of RPE cells exhibiting confluent hexagonal cells, pigmentation, cobblestone configuration, and well-defined membrane borders. In some embodiments, a culture of the preparation comprises about 95% (e.g., about 96%, 97%, 98%, 99% or more) of RPE cells exhibiting confluent hexagonal cells, pigmentation, cobblestone configuration, well-defined membrane borders, and polarization (e.g., as assessed using Ezrin staining).

In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and/or (d) at least 50% of the RPE cells exhibit ability to phagocytose photoreceptor outer segments, e.g., as measured by flow cytometry. In some embodiments, (a) the RPE cells exhibit trans-epithelial electrical resistance (TEER) of at least 100 ohms/cm² after culturing for about 1 week to about 8 weeks; (b) the RPE cells exhibit differential secretion of VEGF at a basal to apical secretion ratio greater than 0.5; (c) the RPE cells exhibit differential secretion of PEDF at an apical to basal secretion ratio greater than 0.5; and (d) at least 50% of the RPE cells exhibit ability to phagocytose photoreceptor outer segments, e.g., as measured by flow cytometry.

In some embodiments, upon passage through a transplantation device (e.g., a 41 g device), at least 80% (e.g., at least 85%, 90%, 91%, 92%, 93%, 94%, 95% or more) of the RPE cells are viable, e.g., as determined by trypan blue staining. In some embodiments, upon passage through a transplantation device (e.g., a 41 g device), about 80% (e.g., about 85%, 90%, 91%, 92%, 93%, 94%, 95% or more) of the RPE cells are viable, e.g., as determined by trypan blue staining.

In some embodiments, upon passage through a transplantation device (e.g., a 41 g device), at least 80% (e.g., at least 85%, 90%, 91%, 92%, 93%, 94%, 95% or more) of the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture. In some embodiments, upon passage through a transplantation device (e.g., a 41 g device), about 80% (e.g., about 85%, 90%, 91%, 92%, 93%, 94%, 95% or more) of the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture.

In some embodiments, upon storage at room temperature for about 6 hours, at least 80% (e.g., at least 85%, 90%, 91%, 92%, 93%, 94%, 95% or more) of the RPE cells are viable, e.g., as determined by trypan blue staining. In some embodiments, upon storage at room temperature for about 6 hours, about 80% (e.g., about 85%, 90%, 91%, 92%, 93%, 94%, 95% or more) of the RPE cells are viable, e.g., as determined by trypan blue staining.

In some embodiments, the iPSCs are derived from fibroblasts or from peripheral blood mononuclear cells.

In some embodiments, the preparation comprises a suspension of the RPE cells. In some embodiments, the preparation comprises a scaffold, matrix, or bio ink, wherein the RPE cells are seeded onto, cultured onto, printed onto, or embedded in, the scaffold, matrix, or bio ink.

In some embodiments, the preparation comprises about 10,000 to about 1,500,000 RPE cells.

Other features, objects, and advantages of the present disclosure are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### DEFINITIONS

A or An: The articles “a” and “an” are used herein to refer to one or to more than one (i.e., to at least one) of the grammatical object of the article. By way of example, “an element” means one element or more than one element.

Administration: As used herein, the term “administration” typically refers to the administration (e.g., of a composition or treatment) to a subject or system (e.g., that is or comprises one or more cells, tissues, organisms, etc.), for example to achieve delivery of an agent that is, is included in, or is otherwise delivered or generated by, such composition or treatment.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biodegradable: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid) (PLA), poly(glycolic acid) (PGA), poly(lactic-co-glycolic acid) (PLGA), and copolymers with PEG, polyanhydrides, poly(ortho) esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, extra cellular matrix (ECM) derived scaffolds, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Differentiation: The term “differentiation” as used herein is the process by which an unspecialized (“uncommitted”) or less specialized cell acquires the features of a specialized cell such as, for example, an RPE cell. In some embodiments, a differentiated or differentiation-induced cell is one that has taken on a more specialized (“committed”) position within the lineage of a cell. For example, an iPSC can be differentiated into various more differentiated cell types, for example, a neural or a hematopoietic stem cell, a lymphocyte, a cardiomyocyte, and other cell types, upon treatment with suitable differentiation factors in the cell culture medium. In some embodiments, suitable methods, differentiation factors, and cell culture media for the differentiation of pluri- and multipotent cell types into more differentiated cell types are well known to those of skill in the art. In some embodiments, the term “committed”, is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type.

Differentiation marker: The terms “differentiation marker”, “differentiation marker gene”, or “differentiation gene”, as used herein refers to genes or proteins whose expression are indicative of cell differentiation occurring within a cell, such as a pluripotent cell. In some embodiments, differentiation marker genes include, but are not limited to, the following genes: RPE65, CRALBP, PEDF, Bestrophin-1 (BEST1), RLBP1, merTK, MITF, OTX2, PAX2, PAX6, premelanosome protein (gp-100 or PMEL, e.g., PMEL-17), CD140b, tyrosinase, and/or ZO1.

Expression: As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Induced pluripotent stem cell: The terms “induced pluripotent stem cell” or “iPSC” as used herein to refer to a stem cell obtained from a differentiated somatic (e.g., adult, neonatal, or fetal) cell by a process referred to as reprogramming (e.g., dedifferentiation). In some embodiments, reprogrammed cells are capable of differentiating into tissues of all three germ or dermal layers: mesoderm, endoderm, and ectoderm. iPSCs are not found in nature.

Marker: A marker, as used herein, refers to an entity or moiety whose presence or level is a characteristic of a particular state or event. In some embodiments, presence or level of a particular marker may be characteristic of a particular type of cell or cell stage.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population.

Prevent: The terms “prevent”, “preventing”, and “prevention” as used herein in the context of a disease refer to the prevention of the disease in a mammal, e.g., in a human, including (a) avoiding or precluding the disease; (b) affecting the predisposition toward the disease; or (c) preventing or delaying the onset of at least one symptom of the disease.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Reprogramming: The terms “reprogramming” or “dedifferentiation” as used herein refer to a method of increasing potency of a cell or dedifferentiating a cell to a less differentiated state. For example, in some embodiments, a cell that has an increased cell potency has more developmental plasticity (i.e., can differentiate into more cell types) compared to the same cell in the non-reprogrammed state. That is, in some embodiments, a reprogrammed cell is one that is in a less differentiated state than the same cell in a non-reprogrammed state. In some embodiments, “reprogramming” refers to de-differentiating a somatic cell, or a multipotent stem cell, into a pluripotent stem cell, also referred to as an induced pluripotent stem cell, or iPSC. Suitable methods for the generation of iPSCs from somatic or multipotent stem cells are well known to those of skill in the art.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Suffering from: An individual who is “suffering from” a disease, disorder, and/or condition displays one or more symptoms of a disease, disorder, and/or condition and/or has been diagnosed with the disease, disorder, or condition.

Therapeutically effective amount: As used herein, the term “therapeutically effective amount” refers to an amount of a preparation of RPE cells or composition described herein that confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a preparation of RPE cells or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular a preparation of RPE cells or composition, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism; the duration of the treatment; and like factors as is well known in the medical arts.

Treatment: As used herein, the term “treatment” (also “treat” or “treating”) refers to any administration of a preparation of RPE cells or composition described herein that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF DRAWINGS

FIG. 1 shows a flow diagram of an exemplary AiPSC-RPE manufacturing process.

FIG. 2A shows steps 1 and 2 of an exemplary morphology scoring system. FIG. 2B shows steps 3 and 4 of an exemplary morphology scoring system.

FIG. 3A shows viability of AiPSC-RPE cell product held in final formulation for 0, 2, 4, and 6 hours versus freshly thawed AiPSC-RPE cell products. AiPSC-RPE cell products that were cultured before preparing the final formulation had higher viability. FIG. 3B shows potency measured by TEER for AiPSC-RPE cell products that were freshly thawed, 1 week cultured or 4 weeks in culture. AiPSC-RPE cell products that were cultured before preparing the final formulation had greater Ohm*cm².

FIG. 4 shows viability in final formulation up to 6 hours of AiPSC-RPE final products (AiPSC-RPE that were cultured before prepared in final formulation) that were passed through the delivery device. Viability was greater than 90%. Lonza RPE is positive control.

FIG. 5A shows differential secretion of cytokine VEGF of AiPSC-RPE final products that were passed through the delivery device. FIG. 5B shows differential secretion of cytokine PEDF of AiPSC-RPE products that were passed through the delivery device. A ratio of over one is indicative of healthy RPE cells. Lonza RPE is positive control.

FIG. 6A shows % of AiPSC-RPE products expressing PMEL17, as measured by flow cytometry. FIG. 6B shows % of AiPSC-RPE products expressing CD140b, as measured by flow cytometry. FIG. 6C shows % of AiPSC-RPE products expressing GD2, as measured by flow cytometry. FIG. 6D shows % of AiPSC-RPE products expressing CD184, as measured by flow cytometry.

FIG. 7 is a schematic of an exemplary method of assessing parallel cultures from a representative RPE bank.

FIG. 8 shows thickness of outer nuclear layer (ONL) as assessed from OCT images in RCS rats for 4, 8, and 12 weeks post-injection. Vehicle control animals were treated with BSS+ solution only, untreated animals were not injected, AiPSC-RPE cell line 1: 01F1i1R1, AiPSC-RPE cell line 2: 02F1i1R1, AiPSC-RPE cell line 3: 13F1i1R1 and cell line 4: 13F1i2R1. N=6 RCS rats, mean±stdev.

FIG. 9 shows a panel of images of stained retina cryo sections from 4 weeks post transplantation with AiPSC-RPE cell line 01F1i1R1, imaged at 20×. Top panel from left to right, STEM101 (human nuclear), RPE65 (RPE cell mature marker) and DAPI (control nuclear), and merge image. Bottom panel shows secondary antibody only controls for STEM101, RPE65, RLBP1 and DAPI. Images show persistence of AiPSC-RPE at 4 weeks post implantation.

FIG. 10 shows a panel of images of stained retina cryo sections from 12 weeks post transplantation with AiPSC-RPE cell line 02F1i1R1, imaged at 63×. Top panel from left to right, RPE65, TRA-1-85 (Human cell surface antigen), DAPI (nuclear stain) and merge. Bottom panel shows secondary antibody only controls for DAPI, RPE65, TRA-1-85. Images show persistence of AiPSC-RPE at 12 weeks post transplantation.

FIG. 11 shows an image of stained retina cryo sections from 12 weeks post transplantation with AiPSC-RPE cell line 02F1i1R1 and imaged at 5×. Merge image, DAPI, RPE65, TRA-1-85 (Human cell surface antigen). The image shows migration and integration of AiPSC-RPE from the injection site throughout the retina.

FIG. 12 shows optokinetic gain measured in RCS rats at days 24, 54 and 88. Vehicle control animals were injected with BSS+ solution only, untreated animals were not injected, AiPSC-RPE cell lines: 01F1i1R1 and 02F1i1R1.

FIG. 13 shows ERG Scotopic Vision 39.8 cd.see/m² Full Field ERG measured in RCS rats at weeks 4, 8 and 12. Vehicle control animals were injected with BSS+ solution only, untreated animals were not injected, AiPSC-RPE cell lines: 01F1i1R1, 02F1i1R1, 13F1i1R1 and 13F1i2R1.

### DETAILED DESCRIPTION OF CERTAIN EMBODIMENTS

The present disclosure is based, in part, on the discovery of certain characteristics exhibited by RPE cells, e.g., RPE cells differentiated from iPSCs, that are correlated with increased viability, purity, functionality, stability, and phenotype. Certain aspects of the present disclosure relate to use of such characteristics as criteria, e.g., quality control criteria, for preparations of RPE cells to be used as cellular therapeutics to treat subjects having or suffering from eye disorders. The present disclosure is also based, in part, on the discovery of methods of producing preparations of RPE cells that result in RPE cells exhibiting such characteristics, e.g., quality control criteria.

Several degenerative diseases of the eye cause permanent vision loss due to retinal pigment epithelial (RPE) cell dysfunction. Treatments for these diseases represent a major unmet medical need. Clinical trials with human embryonic stem cell (hESC) have been described (Schwartz et al., Lancet 379 (9817): 713-20 (2012); Schwartz et al., Lancet 385 (9967): 509-16 (2015)), as well as published trials using induced pluripotent stem cells (iPSC) derived RPE cells (Mandai et al., N. Engl. J. Med. 376 (11): 1038-1046 (2017)) to treat patients with macular dystrophies. The present disclosure is based, in part, on discovery of new methods for generating patient-specific autologous iPSC-derived RPE cells for transplantation.

In most mammals, including humans, the photoreceptor (PR) layer is responsible for translating light into signals received by the brain. Although the PR layer is crucial for vision, so too are the other layers of the eye, including the RPE which is located between the choroid and the PRs. The healthy RPE is organized in a polarized monolayer with tight junctions, helps maintain PR function by recycling photo pigments, phagocytosing PR outer segments, transporting ions and small molecules between retina and choroid, maintaining Bruch's membrane and absorbing stray light to allow better image resolution. Therefore, the RPE layer is critical to the function and health of PRs. (Strauss, Physiol. Rev. 85 (3): 845-81 (2005)).

Unfortunately, many diseases include the loss of the RPE and PR cells in the eye, resulting in blindness. Diseases such as age-related macular degeneration (AMD), Stargardt macular dystrophy (SMD), and retinitis pigmentosa (RP) feature an initial period of cellular dysfunction followed by cell loss, including RPEs, that ultimately causes permanent blindness. In addition, other disorders such as Best disease and macular telangiectasia can result in RPE and PR loss. (Bitner et al., Am. J. Ophthalmol. 154 (2): 403-412.e4 (2012)). Although these diseases feature disparate underlying disease processes, the endpoint is the same: loss of the RPE and eventual blindness.

There is an unmet medical need for therapeutics to reconstitute the RPE and PRs. Transplantation of replacement RPE cells represents an attractive therapeutic paradigm because it addresses a common endpoint for all these conditions. Using patient-specific iPSC-derived RPEs offers further advantages because transplanted cells match the patient precisely. In some embodiments, methods can include harvesting patient skin cells, followed by reprogramming of the cells to a pluripotent state using a combination of transcription factors, (Yang et al., N. Engl. J. Med. 359 (14): 1456-63 (2008)) and then differentiating the iPSC into patient-specific RPE cells for transplantation. Generation of RPE cells and their in vitro and in vivo characterization is well established. (Schwartz et al, Lancet 379 (9817): 713-20 (2012); Abe et al., Curr. Eye Res. 20 (4): 268-75 (2000); Carr et al., PLOS One 4 (12): e8152 (2009); Kamao et al., Stem Cell Reports 2 (2): 205-18 (2014); Lu et al., Stem Cells 27 (9): 2126-35 (2009); Lund et al., Cloning Stem Cells 8 (3): 189-99 (2006)). RPE cells derived from hESC and iPSCs have been shown to share similar morphological and functional characteristics. (Riera et al., Mol. Ther. Methods Clin. Dev. 3:16010 (2016)). Multiple reports on functional outcomes of sub retinal transplantation of RPE cells in animal models of retinal degeneration (Carr et al., PLOS One 4 (12): e8152 (2009); Kamao et al., Stem Cell Reports 2 (2): 205-18 (2014); Li et al., Mol. Med. 18 (1): 1312-9 (2012)) and approved clinical trials for RPE cell replacement therapy have been described. However, there remains a need for effective RPE cellular therapy. Methods and compositions of the present disclosure can be used to address such unmet medical need.

### Methods of Manufacturing Cell Preparations

In some embodiments, methods of the disclosure include providing or producing induced pluripotent stem cells (iPSCs), and subsequently differentiating such iPSCs into RPE cells.

Production of iPSCs

In some embodiments, methods of the disclosure include obtaining and/or producing induced pluripotent stem cells (IPSCs). iPSCs are a type of pluripotent stem cell artificially derived from a non-pluripotent cell, such as an adult somatic cell (e.g., a fibroblast cell or other suitable somatic cell), by inducing expression of certain genes. Various suitable methods for producing iPSCs are known in the art (see, e.g., Takahashi et al., Cell 126:663-676 (2006); Seki et al., World J. Stem Cells 7 (1): 116-125 (2015); and Lakshmipathy and Vermuri, editors, Methods in Molecular Biology: Pluripotent Stem Cells, Methods and Protocols, Springer 2013). In some embodiments, iPSCs can be derived by transfection of certain stem cell-associated genes (such as Oct-¾ (Pouf51) and Sox-2) into non-pluripotent cells, such as adult fibroblasts. In some instances, transfection can be achieved through viral vectors, such as retroviruses, lentiviruses, or adenoviruses. Additional suitable reprogramming methods include the use of vectors that do not integrate into the genome of the host cell, e.g., episomal vectors, or the delivery of reprogramming factors directly via encoding RNA or as proteins has also been described. For example, cells can be transfected with Oct-¾, Sox-2, Klf4, and/or c-Myc using a retroviral system or with Oct-4, Sox-2, NANOG, and/or LIN28 using a lentiviral system. In some embodiments, reprogramming can be induced by the non-viral introduction of reprogramming factors, e.g., by introducing the proteins themselves, or by introducing nucleic acids that encode the reprogramming factors, for example by introducing messenger RNAs that upon translation produce the reprogramming factors (see e.g., Warren et al., Cell Stem Cell, 2010 Nov. 5; 7 (5): 618-30). Numerous suitable methods for reprogramming are known to those of skill in the art, and the present disclosure is not limited in this respect. Additional methods and systems are commercially available (e.g., Simplicon® RNA Reprogramming Kit, EMD Millipore).

In some embodiments, iPSCs can be derived from somatic cells. Somatic cells, as that term is used herein, refer to cells forming the body of an organism, excluding germline cells. Every cell type in the mammalian body (apart from the sperm and ova, the cells from which they are made (gametocytes) and undifferentiated stem cells) is a differentiated somatic cell. For example, internal organs, skin, bones, blood, and connective tissue are all made up of differentiated somatic cells. In some embodiments, mature cells from which iPSCs are made include somatic cells; in some embodiments, such somatic cells may be or comprise blood cells, e.g., blood mononuclear cells such as whole blood mononuclear cells or peripheral blood mononuclear cells (PBMCs). In some embodiments, mature cells from which iPSCs are made may be or comprise B lymphocytes (B-cells), T lymphocytes, (T-cells), fibroblasts, keratinocytes, etc.

In some embodiments, appropriate somatic cell types for use in accordance with the present disclosure include: fibroblasts (e.g., primary fibroblasts), muscle cells (e.g., myocytes), cumulus cells, neural cells, mammary cells, hepatocytes and pancreatic islet cells. In some embodiments, a somatic cell is a primary cell line or is the progeny of a primary or secondary cell line. In some embodiments, a somatic cell is obtained from a human sample, e.g., a hair follicle, a blood sample, a biopsy (e.g., a skin biopsy or an adipose biopsy), a swab sample (e.g., an oral swab sample), etc., and is thus a human somatic cell.

Some non-limiting examples of differentiated somatic cells include, for instance, epithelial, endothelial, neuronal, adipose, cardiac, skeletal muscle, skin, immune cells, hepatic, splenic, lung, peripheral circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, blood, etc. In various embodiments, a somatic cell can be from a mammalian species, with non-limiting examples including a murine, bovine, simian, porcine, equine, ovine, or human. In some embodiments, the somatic cell is a human somatic cell. In preferred embodiments, the somatic cell is an autologous human somatic cell.

Reprogrammed somatic cells can express any number of pluripotent cell markers, such as, for example: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; beta-Ill-tubulin; alpha-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct¾; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4. Other markers can include Dnmt3L; Sox15; Stat3; Grb2; β-catenin, and Bmi1.

In some embodiments, iPSCs are derived from human fibroblasts, e.g., from a human skin biopsy. Methods of producing iPSCs from human fibroblasts are known in the art (e.g., Hazim et al., Stem Cell Res. Ther. 8 (1): 217 (2017); Hazim et al., Stem Cell Res. Ther. 10 (1): 52 (2019)). In some embodiments, human fibroblasts are isolated from a human skin biopsy, expanded in cell culture, and characterized for expression of one or more fibroblast cell markers (e.g., Fibroblast Specific Protein-1 (FSP-1) and/or Vimentin (VIM)), e.g., by immunostaining. In some embodiments, cultured fibroblasts are selected for one or more further processing steps (e.g., cryopreserving and/or reprogramming into iPSCs) if they are double positive for FSP-1 and VIM. In some embodiments, fibroblasts are selected for cryopreservation if they are double positive for FSP-1 and VIM. In some embodiments, cryopreserved fibroblasts are thawed before reprogramming into iPSCs, e.g., using a method described herein. In some embodiments, iPSCs are characterized for expression of one or more pluripotent cell markers, e.g., one or more pluripotent cell markers described herein, e.g., SSEA-4 and/or Oct-4. In some embodiments, iPSCs are selected for one or more further processing steps (e.g., cryopreserving and/or differentiation into RPE cells as described herein) if they express one or more pluripotent cell markers, e.g., one or more pluripotent cell markers described herein, e.g., if they are double positive for SSEA-4 and Oct-4.

### Production of RPE Cell Preparation

In some embodiments, methods of the disclosure include producing RPE cells, e.g., differentiating iPSCs into RPE cells. Certain methods of differentiating iPSCs into RPE cells are known in the art (e.g., Hazim et al., Stem Cell Res. Ther. 8 (1): 217 (2017); Hazim et al., Stem Cell Res. Ther. 10 (1): 52 (2019)). In some embodiments, differentiation to RPE cells is initiated by replacement of culture medium that supports pluripotency with a differentiation medium, e.g., a medium that includes one or more differentiation-inducing factors such as activin, a nodal signal inhibitor, a Wnt signal inhibitor, and/or a sonic hedgehog signal inhibitor.

In some embodiments, a differentiation medium comprises Dulbecco's Modified Eagle Medium: Nutrient Mixture F-12 (DMEM/F12) with xeno-free knockout serum (Invitrogen), NEAA, glutamine, and/or nicotinamide. In some embodiments, a differentiation medium may include one or more additional differentiation agents, such as a member of the transforming factor-β (TGFβ) superfamily (e.g., activin A, activin B, and activin AB), nodal, anti-mullerian hormone (AMH), bone morphogenetic proteins (BMP) (e.g., BMP2, BMP3, BMP4, BMP5, BMP6, and BMP7, growth and differentiation factors (GDF)), WNT pathway inhibitor (e.g., CKI-7, DKK1), a TGF pathway inhibitor (e.g., LDN193189, Noggin), a BMP pathway inhibitor (e.g., SB431542), a sonic hedgehog signal inhibitor, a bFGF inhibitor, and/or a MEK inhibitor (e.g., PD0325901).

In some embodiments, iPSCs are differentiated to an RPE cell lineage in a first differentiation medium comprising a first differentiation agent and then further differentiated to RPE cells in a second differentiation medium comprising a second differentiation agent. In some embodiments, a first differentiation medium comprises nicotinamide (e.g., about 5 mM nicotinamide to about 20 mM nicotinamide, e.g., about 10 mM nicotinamide) and a second differentiation medium comprises activin (e.g., activin A) (e.g., about 100 ng/ml Activin A to about 300 ng/ml Activin A, e.g., about 140 ng/ml Activin A). In some embodiments, a first differentiation medium comprises DMEM/F12 supplemented with about 10% to about 20% (e.g., about 14%) xeno-free knockout serum, about 0.05 mM to about 5 mM (e.g., about 0.1 mM) NEAA, about 0.5 mM to about 5 mM (e.g., about 2 mM) L-glutamine, and about 5 mM to about 20 mM (e.g., about 10 mM) nicotinamide; and a second differentiation medium comprises DMEM/F12 with Activin A (e.g., about 100 ng/ml Activin A to about 300 ng/ml Activin A, e.g., about 140 ng/ml Activin A) and about 5 ng/ml to about 50 ng/ml (e.g., about 20 ng/ml) basic fibroblast growth factor (e.g., FGF2). In some embodiments, differentiation media is changed every day or every 2-3 days during differentiation (e.g., in a first differentiation medium and/or a second differentiation medium). In some embodiments, cells are cultured in a first differentiation medium and/or a second differentiation medium for about 3-12 weeks, e.g., 6-10 weeks, 2-8 weeks, or 3-6 weeks. In some embodiments, cells are cultured in a first differentiation medium for about 2 weeks and then cultured in a second differentiation medium for about 2 weeks.

In some embodiments, after cells are cultured in a first and/or a second differentiation medium, cells are subsequently cultured in DMEM/F12 basal medium, e.g., until pigmentation is detected (e.g., until a majority of cells, e.g., about 50%, 60% 70%, 80%, 85%, 90%, 95%, 97% or more cells, are determined to be pigmented, e.g., using visual analysis such as using a phase contrast microscope). In some embodiments, pigmented cells are differentially passaged, e.g., with a dissociation reagent (e.g., trypsin, e.g., TrypLE) and RPE medium containing DMEM/F12 supplemented with Fetal Bovine Serum (FBS) (e.g., about 2% to about 8%, e.g., about 5% FBS), human AB serum (e.g., about 2% to about 6%, e.g., about 4% human AB serum), taurine (e.g., about 0.05 to about 0.5 mg/ml, e.g., about 0.25 mg/ml taurine), nicotinamide (e.g., about 5 mM to about 20 mM, e.g., 10 mM nicotinamide), NEAA, N2, B27, beta mercaptoethanol (e.g., about 0.05 mM to about 0.5 mM, e.g., about 0.1 mM beta mercaptoethanol) and 1× Glutamax™. In some embodiments, pigmented cells are passaged mechanically by collecting cells with medium after about 5, 8, 10, 12, or about 15 minutes of TrypLE treatment. In some embodiments, cells are differentially passaged at least once, at least twice, at least 3 times, at least 4 times, at least 5 times, at least 6 times, or at least 8 times.

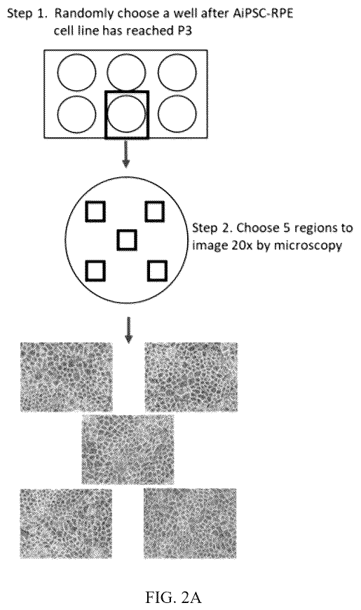

In some embodiments, one or more morphology quality control criteria, e.g., presence of one or more of the following morphology parameters, are assessed to determine the timing for one or more passaging steps (e.g., the duration of treatment with dissociation reagent, e.g., trypsin treatment) and/or the number of passaging steps: confluent hexagonal cells, pigmentation, cobblestone configuration, and/or well-defined membrane borders (see, e.g., FIG. 2). In some embodiments, after one passaging step, cells are assessed for one or more morphology parameters.

In some embodiments, if after one passaging step, cells do not meet morphology quality control criteria (e.g., at least 80%, 85%, 90%, or 95% of cells do not exhibit one or more morphology parameters), the cells are subjected to at least one additional passaging step. In some embodiments, if after one passaging step, cells do not meet morphology quality control criteria (e.g., at least 80%, 85%, 90%, or 95% of cells do not exhibit all morphology parameters), the cells are subjected to at least one additional passaging step. In some embodiments, passaging steps are repeated until morphology quality control criteria are met (e.g., at least 80%, 85%, 90%, or 95% of cells exhibit one or more morphology parameters). In some embodiments, passaging steps are repeated until morphology quality control criteria are met (e.g., at least 80%, 85%, 90%, or 95% of cells exhibit all morphology parameters).

In some embodiments, cells that have been passaged as described herein and meet such morphology quality control criteria, also meet one or more of the following functional quality control criteria: (i) the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture; (ii) the cells exhibit differential secretion of VEGF at a basal to apical secretion ratio of at least about 0.25, at least about 0.5, at least about 0.75, at least about 1, at least about 1.25, or at least about 1.5; (iii) the cells exhibit differential secretion of PEDF at an apical to basal secretion ratio of at least about 0.25, at least about 0.5, at least about 0.75, at least about 1, at least about 1.25, or at least about 1.5; and/or (iv) at least about 50%, about 60%, about 70%, about 80%, about 90%, or about 95% of the RPE cells exhibit ability to phagocytose POR (e.g., as assessed using flow cytometry).

In some embodiments, RPE cells that meet one or more such quality control criteria (e.g., one or more morphology quality control criteria described herein and/or one or more functional quality control criteria described herein) are selected for one or more further processing steps (e.g., expansion, formulation into a preparation of RPE cells, e.g., RPE cell drug substance, and/or cryopreserving). In some embodiments, RPE cells that meet all of the morphology quality control criteria described herein and all of the functional quality control criteria described herein are selected for one or more further processing steps (e.g., expanding to achieve a predetermined number of cells, formulating into a preparation of RPE cells, e.g., formulating as RPE cell drug substance, and/or cryopreserving). In some embodiments, RPE cells that meet all of the morphology quality control criteria described herein and all of the functional quality control criteria described herein are expanded to achieve a predetermined number of cells, are formulated into a preparation of RPE cells (e.g., RPE cell drug substance) and are cryopreserved.

### Production of RPE Cell Product

In some embodiments, methods of the disclosure include producing an RPE cell product (e.g., RPE cell drug product) from an RPE cell preparation (e.g., RPE cell drug substance) produced, e.g., as described herein. In some embodiments, an RPE cell preparation (e.g., RPE cell drug substance) has been subjected to/has undergone at least one freeze/thaw cycle. Certain aspects of the disclosure are based on the discovery that culturing such previously cryopreserved and frozen RPE cell preparations, e.g., prior to therapeutic formulation and/or administration to a subject, is beneficial. For example, culturing such previously cryopreserved and frozen RPE cell preparations can improve viability and/or potency of such cells. In some embodiments, a frozen RPE cell preparation (e.g., frozen RPE cell drug substance) is thawed, and the cells are cultured in appropriate cell culture medium for about 1 week, about 2 weeks, about 3 weeks, about 4 weeks, about 5 weeks, or about 6 weeks. In some embodiments, following such culturing step, RPE cells have at least about 80%, 85%, 90%, 95%, viability, e.g., relative to a reference preparation of RPE cells, e.g., RPE cell drug substance described herein, that has not undergone at least one freeze/thaw cycle. In some embodiments, following such culturing step, at least 80%, 85%, 90%, 95%, or more of the RPE cells are viable. In some embodiments, such cultured RPE cells are formulated as a therapeutic composition, e.g., an RPE cell drug product.

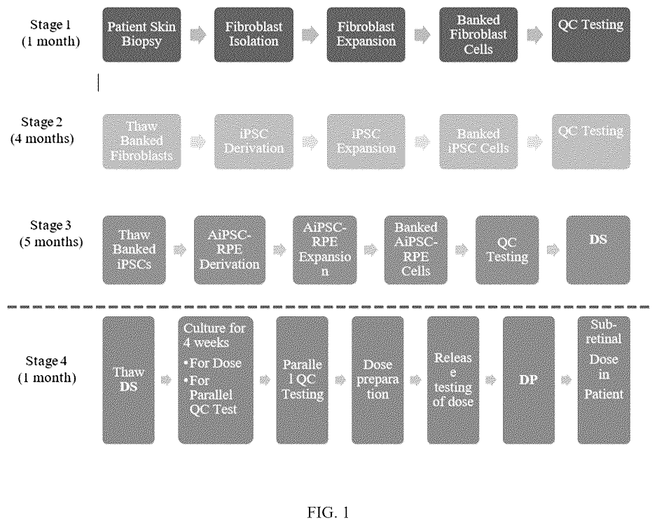

In some embodiments, methods of producing an RPE cell drug product can include four stages, e.g., as depicted schematically in FIG. 1. As shown in FIG. 1, stage 1 can include steps of isolating fibroblasts from a subject's skin biopsy; expanding isolated fibroblasts; banking fibroblasts; and optionally testing banked fibroblasts as described herein. As shown in FIG. 1, stage 2 can include steps of thawing banked fibroblasts; reprogramming fibroblasts to iPSCs; expanding iPSCs; banking iPSCs; and optionally testing banked iPSCs using one or more criteria as described herein. As shown in FIG. 1, stage 3 can include steps of thawing banked iPSCs; differentiating iPSCs into RPE cells; expanding RPE cells; banking RPE cells (e.g., as RPE cell drug substance (DS)); and optionally testing banked RPE cells using one or more criteria described herein. As shown in FIG. 1, stage 4 can include steps of thawing banked RPE cells (DS); culturing thawed RPE cells for about 4 weeks; testing RPE cells using one or more criteria described herein; preparing dose of preparation of RPE cells; release testing of dose using one or more criteria described herein; and producing RPE cell drug product (DP). The durations of various stages depicted in FIG. 1 are exemplary, and in some embodiments, the duration of one or more stages depicted in FIG. 1 can be shorter or longer relative to the times depicted in FIG. 1. For example, while the exemplary method of FIG. 1 notes a duration of stage 1 of about 1 month, in some embodiments, stage 1 can be shorter (e.g., about 1, 2, 3, or 3½ weeks), or can be longer (e.g., about 4½, 5, 6, 7, or 8 weeks). Additionally or alternatively, while the exemplary method of FIG. 1 notes a duration of stage 2 of about 4 months, in some embodiments, stage 2 can be shorter (e.g., about 1, 2, 3, or 3½ months), or can be longer (e.g., about 4½, 5, 6, 7, or 8 months). Additionally or alternatively, while the exemplary method of FIG. 1 notes a duration of stage 3 of about 5 months, in some embodiments, stage 3 can be shorter (e.g., about 1, 2, 3, 4, or 4½ months), or can be longer (e.g., about 5½, 6, 7, or 8 months). Additionally or alternatively, while the exemplary method of FIG. 1 notes a duration of stage 4 of about 1 month, in some embodiments, stage 4 can be shorter (e.g., about 1, 2, 3, or 3½ weeks), or can be longer (e.g., about 4½, 5, 6, 7, or 8 weeks). In some embodiments, the duration of one or more stages depicted in FIG. 1 is determined using quality control criteria (e.g., morphology parameters and/or functional parameters) described herein.

### Methods of Assessing Cells

In some embodiments, methods of the disclosure include assessing cells, e.g., iPSCs and/or RPE cells described herein. In some embodiments, cells can be assessed for expression of one or more pluripotent cell markers, including, e.g., alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; beta-Ill-tubulin; alpha-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct¾; Sox2; Klf4; Lin28, c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; and/or DPPA4. Other markers can include Dnmt3L; Sox15; Stat3; Grb2; β-catenin, and/or Bmi1. In some embodiments, cells can be assessed for expression of one or more RPE cell markers, such as RPE65, CRALBP, PEDF, Bestrophin-1 (BEST1), RLBP1, merTK, MITF, OTX2, PAX2, PAX6, premelanosome protein (gp-100 or PMEL, e.g., PMEL-17), CD140b, tyrosinase, and/or ZO1. In some embodiments, cells can be assessed for expression of non-RPE linage markers, such as GD2 and/or CD184. Methods of assessing expression of such markers are known in the art, such as flow cytometry, immunohistochemistry, PCR, ddPCR, and next generation sequencing.

In some embodiments, cells are assessed for presence of one or more morphological characteristics, such as morphology parameters described herein. Methods of assessing such morphological characteristics are known in the art, such as visual assessment using microscopy. In some embodiments, cells are assessed for viability using methods known in the art, e.g., using trypan blue staining.

In some embodiments, cells are assessed for one or more functional parameters, such as functional parameters described herein. For example, transepithelial electrical resistance (TEER) can be measured, e.g., to assess barrier function, using methods known in the art (see, e.g., Markert et al., Front. Cell Dev. Biol. 10:910040 (2022); Hazim et al., Stem Cell Res. Ther. 8 (1): 217 (2017)). Devices for measuring TEER are known in the art and include, for example, EVOM2 Epithelial Voltohmmeter (World Precision Instruments). In some embodiments, potency of cells is assessed by analyzing polarized PEDF and VEGF secretion, e.g., using methods known in the art. For example, RPE cells can be cultured onto matrigel-coated transwell membranes, apical- and basal-side media can be collected, and PEDF and VEGF measured by ELISA (see, e.g., Zhang et al., Stem Cell Res. Ther. 13 (1): 454 (2022)). Ability of RPE cells to phagocytose photoreceptor outer segments can be measured using methods known in the art (see, e.g., Hazim et al., Stem Cell Res. Ther. 8 (1): 217 (2017)).

### RPE Cell Preparations

In some embodiments, the disclosure provides preparations of RPE cells, e.g., RPE cell drug substance and/or RPE cell drug product, that meet certain criteria and/or are defined by certain characteristics. In some embodiments, a preparation of RPE cells, e.g., RPE cell drug substance and/or RPE cell drug product, meets one or more of the following criteria: (1) at least 80%, 85%, 90%, 95%, or 98% of RPE cells exhibit confluent hexagonal cells, pigmentation, cobblestone configuration, and well-defined membrane borders; (2) gene expression of RPE65≥ 0.5, as assessed using ddPCR; (3) gene expression of BEST1≥6, as assessed using ddPCR; (4) gene expression of RLBP≥1, as assessed using ddPCR; (5) gene expression of MerTK>0.15, as assessed using ddPCR; (6) expression of MiTF, BEST1, and/or ZO-1 at same level as, or differs by no more than about 1%, 5%, or 10% from, expression of MITF, BEST1, and/or ZO-1 in a primary RPE cell line; (7) at least about 70%, 80%, 85%, or 90% of cells are viable for up to about 6 hours, e.g., after passage through a transplantation device (e.g., a 41 g device); (8) at least about 70%, 80%, 85%, or 90% of cells express PMEL17; (9) less than about 0.005%, less than 0.004%, less than 0.003%, or less than 0.002% of cells are positive for Lin28, Oct-4, and/or Klf4 expression, e.g., as assessed by ddPCR; (10) at least about 80%, 85%, or 90% of cells express PMEL17 and CD140b but do not express CD184 or GD2 (PMEL17+CD140b+CD184-GD2-); (11) the RPE cells exhibit TEER of at least about 50 ohms/cm², at least about 75 ohms/cm², at least about 100 ohms/cm², at least about 125 ohms/cm², at least about 150 ohms/cm², at least about 175 ohms/cm², or at least about 200 ohms/cm², e.g., at between 1 and 8 weeks in culture; (12) the cells exhibit differential secretion of VEGF at a basal to apical secretion ratio of at least about 0.25, at least about 0.5, at least about 0.75, at least about 1, at least about 1.25, or at least about 1.5; (13) the cells exhibit differential secretion of PEDF at an apical to basal secretion ratio of at least about 0.25, at least about 0.5, at least about 0.75, at least about 1, at least about 1.25, or at least about 1.5; and/or (14) at least about 50%, about 60%, about 70%, about 80%, about 90%, or about 95% of the RPE cells exhibit ability to phagocytose POR (e.g., as assessed using flow cytometry). In some embodiments, a preparation of RPE cells, e.g., RPE cell drug substance and/or RPE cell drug product, meets at least 2, at least 3, at least 4, at least 5, at least 6, at least 7, at least 8, at least 9, at least 10, at least 11, at least 12, at least 13, or all 14 of these criteria. In some embodiments, a preparation of RPE cells, e.g., RPE cell drug substance and/or RPE cell drug product, has undergone at least 1 freeze/thaw cycle and meets at least 2, at least 3, at least 4, at least 5, at least 6, at least 7, at least 8, at least 9, at least 10, at least 11, at least 12, at least 13, or all 14 of these criteria. In some embodiments, a preparation of RPE cells, e.g., RPE cell drug substance and/or RPE cell drug product, has undergone at least 1 freeze/thaw cycle, has been subsequently cultured for about 1, 2, 3, 4 weeks or longer, and meets at least 2, at least 3, at least 4, at least 5, at least 6, at least 7, at least 8, at least 9, at least 10, at least 11, at least 12, at least 13, or all 14 of these criteria.

In some embodiments, methods of the disclosure have a variety of applications and include, e.g., quality control at different stages of manufacture, analysis of RPE cell preparations prior to or after completion of manufacture (e.g., prior to or after distribution to a fill/finish environment or facility), prior to or after release into commerce (e.g., before distribution to a pharmacy, a caregiver, a patient, or other end-user). Thus, a preparation can be any preparation that potentially comprises an RPE cell drug substance or RPE cell drug product. In some embodiments, a preparation is from a stage of manufacture or use that is prior to release to care givers or other end-users; prior to packaging into individual dosage forms, such as syringes, pens, vials, or multi-dose vials; prior to determination that the batch can be commercially released, prior to production of a Certificate of Testing, Material Safety Data Sheet (MSDS) or Certificate of Analysis (CofA) of the preparation. In some embodiments, an RPE cell preparation is from an intermediate step in production, e.g., during a stage of differentiation from iPSCs, during a stage of RPE cell expansion, or during a stage of RPE cell drug product production. In some embodiments, an RPE cell preparation is from stage 3 or stage 4, as depicted in FIG. 1.

Evaluations from methods of the disclosure are useful, e.g., for guiding, controlling or implementing a number of activities or steps in the process of making, distributing, and monitoring and providing for the safe and efficacious use of an RPE cell drug substance and/or RPE cell drug product. Thus, in some embodiments, e.g., responsive to the evaluation, e.g., depending on whether a criterion is met, a decision or step is taken. The method can further comprise one or both of the decision to take the step and/or carrying out the step itself. E.g., the step can comprise one in which the preparation (or another preparation for which the preparation is representative) is: classified; selected; accepted or discarded; released or processed into a drug product; rendered unusable for commercial release, e.g., by labeling it, sequestering it, or destroying it; passed on to a subsequent step in manufacture; reprocessed (e.g., the preparation may undergo a repetition of a previous process step or subjected to a corrective process); formulated, e.g., into drug substance or drug product; combined with another component, e.g., an excipient, buffer or diluent; disposed into a container; divided into smaller aliquots, e.g., unit doses, or multi-dose containers; packaged; shipped; moved to a different location; combined with another element to form a kit; combined, e.g., placed into a package with a delivery device, diluent, or package insert; released into commerce; sold or offered for sale; delivered to a care giver or other end-user; or administered to a subject. E.g., based on the result of the determination or whether one or more criteria are met, or upon comparison to a reference standard, a batch from which a preparation is taken can be processed, e.g., as just described.

Methods described herein may include making a decision: (a) as to whether a preparation may be formulated into drug substance or drug product; (b) as to whether a preparation may be reprocessed (e.g., the preparation may undergo a repetition of a previous process step); or (c) that the preparation is not suitable for formulation into drug substance or drug product. In some instances, the method comprises: formulating as referred to in step (a), reprocessing as referred to in step (b), or rendering the preparation unusable for commercial release, e.g., by labeling it or destroying it, as referred to in step (c).

### Disorders/Diseases

A preparation of RPE cells described herein can be used in cell-based treatments in which RPE cells are needed or would improve treatment. In some embodiments, provided preparations may be used to treat an ocular pathology. In some embodiments, methods of using RPE cells described herein can be used for treating various conditions described in, e.g., U.S. Pat. No. 10,077,424.

The particular treatment regimen, route of administration, and any adjuvant therapy can be tailored in accordance with appropriate medical practice, for example based on particular condition, severity, and a subject's overall health. In some embodiments, administration of RPE cells may be effective to fully restore any vision loss or other symptoms. In some embodiments, administration of RPE cells may be effective to reduce severity of symptoms and/or to prevent further degeneration in a subject's condition. Alternatively or additionally, in some embodiments, RPE cell administration can be used to help treat symptoms of any injury to endogenous RPE layer or to any other ocular epithelial layer. In some embodiments, a preparation of RPE cells described herein can be used to treat age related macular degeneration, myopic degeneration, Stargardt disease, choroidal neovascularization, any condition, or disease that leads to scarring or atrophy or dysfunction of the RPE layer. In some embodiments, a provided preparation is used to treat a condition or disease that leads to retinal atrophy or edema or degeneration or detachment. In some embodiments, a provided preparation is used to treat a condition or disease that leads to loss or dysfunction of photoreceptors. In some embodiments, a provided preparation is used to treat a condition or disease that affects the cornea endothelium or epithelium. In some embodiments, a provided preparation is used to treat an ocular pathology.

In some embodiments, the disclosure provides methods of treating a retinal disease or disorder by administering a preparation of RPE described herein to an eye of a subject. In some embodiments, a retinal disease or disorder includes, for example, retinal degeneration, such as age-related macular degeneration (dry or wet), retinal detachment, retinitis pigmentosa, Stargardt Disease, myopic degeneration, Best's disease and macular telangiectasia.

Age-related macular degeneration (AMD) is a progressive degenerative disease that is the leading cause of vision loss in the elderly population. Degeneration/dysregulation of the retinal pigment epithelium (RPE), a supportive monolayer of cells underlying the photoreceptors, is commonly seen in patients with AMD. While treatment exists for the neovascular/wet form of AMD, there is currently no cure for the non-exudative/dry form of AMD. (Somasundaran S, Constable I J, Mellough C B, Carvalho L S. Retinal pigment epithelium and age-related macular degeneration: A review of major disease mechanisms. Clin Exp Ophthalmol. 2020 Nov;48 (8): 1043-1056. doi: 10.1111/ceo.13834. Epub 2020 Aug. 17. PMID: 32710488; PMCID: PMC7754492.)

In some embodiments, the present disclosure provides methods of treating disorders associated with retinal degeneration, including macular degeneration, by administering to an eye of a subject a preparation of RPE cells described herein. In some embodiments, the present disclosure provides methods of treatment of eye diseases, including hereditary and acquired eye diseases, by administering to an eye of a subject a preparation of RPE cells described herein. Examples of acquired or hereditary eye diseases include age related macular degeneration, myopic degeneration, Stargardt disease, choroidal neovascularization, any condition or disease that leads to scarring or atrophy or dysfunction of the RPE layer, any condition or disease that leads to retinal atrophy or edema or degeneration or detachment, any condition or disease that leads to loss or dysfunction of photoreceptors, any condition or disease that affects the cornea endothelium or epithelium, or any other ocular pathology.

Pathologic myopia represents a subgroup of myopia and affects up to 3% of the world population. Vision loss related to pathologic myopia is of great clinical significance as it can be progressive and/or irreversible, and affects individuals during their most productive years. Progressive retinal pigment epithelial (RPE) thinning and attenuation develops in various clinical stages throughout the fundus. Distribution of RPE atrophy and variable light reflection may be appreciated even in young patients with high myopia. Patients with pathologic myopia present with the presence of myopic maculopathy equal to or more severe than diffuse chorioretinal atrophy. Myopic maculopathy includes diffuse chorioretinal atrophy, patchy chorioretinal atrophy, lacquer cracks, myopic choroidal neovascularization (myopic CNV), and CNV-related macular atrophy. To date, there is no topical, local or systemic pharmacotherapy or surgery that is known to alter effectively the chorioretinal and RPE atrophy. Patients who develop myopic CNV can be treated with anti VEGF agents. (Ryan et al. Retina. 2013, Ohno-Matsui K. Pathologic myopia. Asia Pac J Ophthalmol 2016; 5:415-423).

Stargardt disease is the most common form of juvenile macular degeneration and is characterized by a progressive loss of central vision, with onset of symptoms usually occurring during the teenage years. In affected individuals, mutations are present in the ABCA4 gene, which encodes the adenosine triphosphate (ATP)-binding cassette transporter in photoreceptors. When functional, ABCA4 helps clearing all-trans-retinaldehyde and thus attenuates excess accumulation of the toxic bisretinoid fluorophores that constitute the lipofuscin in the retina. Reduced or absent transporter activity as in STGD1 results in more rapid accumulation of lipofuscin in the retinal pigment epithelium (RPE) cells, the end result of which is RPE and photoreceptor cell degeneration. Currently there is no available treatment to Stragardts disease.

### Formulations and Administration

In some embodiments, RPE cells described herein are delivered or administered to an eye of a subject in a pharmaceutically acceptable ophthalmic formulation, e.g., by intraocular injection or application to the ocular surface. In some embodiments, provided cell preparations are administered subretinally. Concentrations for injections may be at any amount that is effective and non-toxic. In some embodiments, about 10,000 to about 1,000,000 (e.g., about 10,000 to about 5,000) RPE cells are administered to a subject. In some embodiments, about 10,000, 30,000, 50,000, 100,000, 150,000, 200,000, 250,000, 300,000, 350,000, 400,000, 450,000 or 500,000 up to 1,000,000 or more RPE cells are administered to a subject. In some embodiments, cells are administered as a single dose. In some embodiments, cells are administered in multiple doses.

In some embodiments, RPE cells are formulated for delivery in a pharmaceutically acceptable ophthalmic vehicle. In some embodiments, such vehicle maintains a preparation of RPE cells in contact with an ocular surface for a sufficient time period to allow the cells to penetrate the affected regions of the eye, as for example, the anterior chamber, posterior chamber, vitreous body, aqueous humor, vitreous humor, cornea, iris/ciliary, lens, choroid, retina, sclera, suprachoroidal space, conjunctiva, subconjunctival space, episcleral space, intracorneal space, epicorneal space, sub corneal space/corneal endothelial layer, pars plana, surgically-induced avascular regions, the macula, any retinal region. The present disclosure additionally includes products and systems, such as delivery vehicles, comprising RPE cells described herein, as well as kits comprising such delivery vehicles and/or systems.

In some embodiments, preparations of RPE cells described herein are administered to an eye of a subject using an implant or device. In certain embodiments, the device is a biodegradable implant for treating a condition of the eye, for example comprising RPE cells dispersed within a biodegradable polymer matrix. In some embodiments, a device is or comprises particles (e.g., particles that are or comprise a polymer matrix, such as a biodegradable polymer matrix, with which RPE cells as described herein are associated, for example being dispersed therewithin); in some such embodiments, at least about 75% of the particles have a diameter of less than about 10 μm.

In some embodiments, an implant, e.g., biodegradable implant, can be sized for implantation to an ocular region, such as the anterior chamber, the posterior chamber, the vitreous cavity, the choroid, the suprachoroidal space, the conjunctiva, the subconjunctival space, the episcleral space, the intracorneal space, the epicorneal space, subcorneal space/corneal endothelium layer, the sclera, the pars plana, surgically-induced avascular regions, the macula, and the retina/sub retinal space.

In some embodiments, a biodegradable polymer can be or comprise, for example, a poly(lactic-co-glycolic) acid (PLGA) copolymer. In certain embodiments, a ratio of lactic to glycolic acid monomers in the polymer is about 25/75, about 40/60, about 50/50, about 60/40, about 75/25 weight percentage, more preferably about 50/50. Additionally, the PLGA copolymer can be about 20, about 30, about 40, about 50, about 60, about 70, about 80 to about 90 percent by weight of the biodegradable implant. In certain embodiments, a PLGA copolymer can be from about 30 to about 50 percent by weight, preferably about 40 percent by weight of a biodegradable implant.

In some embodiments, preparations of RPE cells described herein are administered to an eye of a subject on a scaffold, matrix, or bio ink.

The volume of a preparation of RPE cells administered according to methods described herein can be dependent on factors such as the mode of administration, number of RPE cells, age of the subject, and type and severity of the disease being treated. In some embodiments, a preparation of RPE cells is administered by injection, e.g., in a liquid volume from about 5.0 microliters to about 50 microliters, from about 50 microliters to about 250 microliters, from about 250 microliters to about 1 milliliter. In some embodiments, RPE cells are administered by injection in a liquid volume of about 150 microliters.

In some embodiments, a preparation of RPE cells is administered to an eye of a subject by intraocular injection. Dosage regimens can be adjusted to provide the optimum desired response (e.g., a therapeutic response).

In some embodiments, a preparation of RPE cells is administered to an eye of a subject one or more times periodically throughout the life of a subject, e.g., once per year, once every 6-12 months, once every 3-6 months, once every 1-3 months, or once every 1-4 weeks. Alternatively, more frequent administration may be desirable for certain conditions or disorders.

In some embodiments, a subject treated with a preparation of RPE cells is also administered immunosuppressive therapy, either before, concurrently with, or after administration of the RPE cells. In some embodiments, e.g., specifically in embodiments in which allogeneic cells are utilized (rather than autologous cells), immunosuppressive therapy may be necessary throughout the life of the subject, or for a shorter period of time. Examples of immunosuppressive therapy include, but are not limited to, one or more of: anti-lymphocyte globulin (ALG) polyclonal antibody, anti-thymocyte globulin (ATG) polyclonal antibody, azathioprine, BASILIXIMAB® (anti-IL-2Ra receptor antibody), cyclosporin (cyclosporin A), DACLIZUMAB® (anti-IL-2Ra receptor antibody), everolimus, mycophenolic acid, RITUXIMAB® (anti-CD20 antibody), sirolimus, tacrolimus (Prograf™), and mycophemolate mofetil (MMF).

The present disclosure includes compositions, e.g., pharmaceutical compositions, containing preparations of RPE cells described herein, formulated together with a pharmaceutically acceptable carrier. Methods well known in the art for making formulations are found, for example, in “Remington: The Science and Practice of Pharmacy” (20th ed., ed. A. R. Gennaro A R., 2000, Lippincott Williams & Wilkins, Philadelphia, Pa.). In some embodiments, RPE cells described herein are formulated with a pharmaceutically acceptable carrier. In some embodiments, pharmaceutical compositions suitable for parental administration can comprise RPE cells in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof. Proper fluidity can be maintained, for example, by the use of coating materials, such as lecithin, by the maintenance of the required particle size in the case of dispersions, and by the use of surfactants.

Prevention of presence of microorganisms can be ensured by inclusion of one or more antibacterial and antifungal agents, for example, paraben, chlorobutanol, phenol sorbic acid, and the like.

Actual dosage levels of RPE cells in pharmaceutical compositions provided by the present disclosure may be varied so as to obtain an amount of RPE cells that is effective to achieve the desired therapeutic response for a particular patient, composition, and mode of administration, without being toxic to the patient. In some embodiments, a selected dosage level will depend upon one or more of a variety of pharmacokinetic factors including the activity of the particular compositions of RPE cells, the route of administration, the time of administration, the duration of the treatment, other drugs, compounds and/or materials used in combination with the particular compositions employed, the age, sex, weight, condition, general health and prior medical history of the patient being treated, and like factors well known in the medical arts.

In some embodiments, therapeutic compositions can be administered with medical devices known in the art. For example, in some embodiments, RPE cells are delivered as a cell suspension using a delivery device (e.g., needle or injection cannula), e.g., having an outer diameter between 90-100 μm. In some embodiments, RPE cells are delivered as a cell suspension using a delivery device (e.g., needle or injection cannula), e.g., having an inner aperture diameter between 65-75 μl. In some embodiments, RPE cells are loaded into a delivery device (e.g., 1 ml syringe) using a 18 G needle. In some embodiments, an 18 G needle may then be replaced with an extension tube (e.g., between 5-10 cm) and air is removed through the extension tube. An injection cannula, e.g., having a tip having an outer diameter between 90-100 μm (e.g., 41 G) may then be attached to the end of the extension tube. In some embodiments, the inner diameter of the aperture of the tip is about 65-75 μm (e.g., about 70 μm). In some embodiments, the cannula comprises a 41 G tip (for example, as manufactured by Peregrine). In some embodiments, the cannula is a 25 G cannula. In some embodiments, the present disclosure provides an article of manufacture comprising an 18 G needle and a 25 G/41 G cannula. Such a device may be used for the uptake of RPE cells and the subsequent intraocular administration of RPE cells. In some embodiments, the device may further comprise an extension tube (e.g., between about 5 and 15 cm) and a syringe (e.g., 1-2 ml syringe). Many other such implants, delivery systems, and modules are known to those skilled in the art. In some embodiments, RPE cells are delivered on/in a scaffold by a designated carrier.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1-AiPSC-RPE Manufacturing Process

Patient-specific autologous iPSC derived RPE (“AiPSC-RPE”) product was manufactured from patient-derived skin biopsies and produced as one batch per patient. The manufacturing process for AiPSC-RPE drug substance (DS) and drug product (DP) comprised the 4 stages presented in FIG. 1, and are discussed below.

### Stage 1 (Skin Biopsy to Fibroblast Bank)

Fibroblasts were produced and banked as described in Hazim et al., Stem Cell Res. Ther. 10 (1): 52 (2019). Briefly, punch biopsies from subjects were obtained and immediately immersed in Dulbecco's Modified Eagle's Medium (DMEM)/F12. The pellets, which contained dissociated cells and tissue clumps, were collected. The medium was changed once every 72 hours until the cell monolayer was 70% confluent, and then the cells were passaged using TrypLE. Once the cells were confluent, they were passaged and characterized by immunostaining for fibroblast cell markers (Fibroblast Specific Protein-1 (FSP-1) and Vimentin) to confirm their identity. Single-cell suspensions of >70% viability were then cryopreserved in ProFreeze chemically defined freeze medium (CDM) per the manufacturer's protocol. A summary of results from characterizing fibroblast cell lines is shown in Table 1.

TABLE 1
Summary Results of Fibroblast characterization

| | | | Result Summary |
|---|---|---|---|
| | | | (n = Donor cell line |
| | Testing | | passed/n = Donor |
| | parameters | Threshold | cell line tested) |
| Identity | Immunostaining | Double positive | 48/48: Double |
| | (Fibroblast | | positive |
| | Specific Protein | | |
| | (FSP), Vimentin | | |
| | (VIM) | | |
| Safety | Endotoxin | Pass = <5.0 EU/mL | 26/26 tested: Pass |
| | USP<85> LAL | | |
| | Mycoplasma | Negative (Neg.) | 48/48: Negative |
| | STR (Short | No contamination | 48/48: No |
| | Tandem Repeats)- | | contamination |
| | test for | | |
| | contamination | | |
| | Karyotyping | Normal | 33/48: Normal |
| | Sterility <USP71> | Negative | 26/26 tested: |
| | | | Negative |

Stage 2 (Fibroblast Bank Thaw to iPSC Bank)

iPSCs were produced and banked as described in Hazim et al., Stem Cell Res. Ther. 10 (1): 52 (2019). Briefly, patient-specific fibroblasts were thawed and cells were then centrifuged. Cells were resuspended in mTESR1 (serum-free and feeder free) media. After an overnight incubation, cells were reprogrammed using the Simplicon reprogramming kit per the manufacturing SOP for a duration of 4-6 weeks.

The non-integrating vector used for reprogramming of fibroblast to iPSCs was the modified, non-infectious self-replicating polycistronic, Venezuelan Equine Encephalitis (VEE) virus RNA replicon RNA system that consistently expresses the reprogramming factors over multiple cell divisions. iPSC colonies formed within 4-6 weeks and cultures were replated using RelesR (Stem Cell technologies, enzyme-free reagent) for propagation. iPSCs were then passaged weekly until passage 7-10, at which point iPSCs were stabilized (approximately 5-7 weeks). MTesR1 media with 1× antibiotic and antimycotic was used for iPSCs, which was filter sterilized using a 0.22 μm filter, stored for 5-7 days at 4 °C, and changed daily. Single-cell suspensions were then cryopreserved in ProFreeze CDM per the manufacturer's protocol. A summary of the results and quality assurance testings is shown in Table 2.

TABLE 2
Summary Results of AiPSC characterization

| | | | Result Summary |
|---|---|---|---|
| | | | (n = Donor cell line |
| | Testing | | passed/n = Donor |
| | parameters | Threshold | cell line tested) |
| Identity | Immunostaining | Double positive | 22/22: Double |
| | Stage Specific | | positive |
| | embryonic | | |
| | antigen-4(SSEA4), | | |
| | Octamer binding | | |
| | transcription | | |
| | factor-4(OCT4) | | |
| Safety | Endotoxin <LAL> | Pass = <5.0 EU/mL | 6/6 tested: Pass |
| | Mycoplasma | Negative (Neg.) | 22/22: Negative |
| | STR (Short | No contamination | 22/22: No |
| | Tandem | | contamination |
| | Repeats)- test for | | |
| | contamination | | |
| | Karyotyping | Normal | 13/22: Normal |
| | Sterility <USP71> | No contamination | 13/13: No |
| | | | contamination |

Stage 3 (iPSC Bank Thaw to RPE Bank)

RPEs were produced and banked as described in Hazim et al., Stem Cell Res. Ther. 10 (1): 52 (2019). Briefly, patient specific iPSCs were thawed in a temperature monitored water bath at 37 °C for 1 minute. Cells were then centrifuged and cultured in MTesR1 media, which was changed daily for 5-7 days until AiPS cells were 80%-95% confluent.

For differentiation to RPE cells, media was switched to basal medium containing DMEM/F12 supplemented with 14% xeno-free knock out serum replacement (KSR), 1% non-essential amino acids (NEAA), 2 mM L-glutamine and 10 mM nicotinamide, and then cultured for 2 weeks. Medium was changed every other day. On the 3rd and 4th weeks of differentiation, 140 ng/ml Activin A and 20 ng/ml basic Fibroblast Growth Factor (FGF) were added to the medium and the cells were allowed to grow for an additional 2 weeks. Cell cultures were then returned to basal medium until visual analysis using phase contrast microscope showed the majority were deeply pigmented. Pigmented regions started to appear during the 5th and 6th weeks of differentiation.

The pigmented regions were differentially passaged with TrypLE and RPE medium containing DMEM/F12 supplemented with 5% Fetal Bovine Serum (FBS), 4% human AB serum, 0.25 mg/ml taurine, 10 mM nicotinamide, 1× NEAA, 1× N2, 1× B27, 0.1 mM beta mercaptoethanol and 1× Glutamax™. Pigmented cells were passaged mechanically by gently collecting the cells with the medium after 10 minutes of TrypLE™ treatment. As shown in Table 3, AiPSC-RPE cells that were passaged using TrypLE for 10 minutes yielded cells that passed identity, safety, stability, purity and potency tests when compared to AiPSC-RPE cells that were passaged using TrypLE for 1 or 3 mins.

TABLE 3
Comparison of differential passaging showing better yields across tests by 10 minutes of
passaging vs 1 or 3 minutes

| | | ddPCR for RPE specific | | | | | | | | |
|---|---|---|---|---|---|---|---|---|---|---|
| | | marker expression (RPE65; | | | | | | | | |
| IHC for RPE | | BEST1; RLBP; MerTK) | | | Microscopy | | Testing | | | |
| PASS: AiPSC-RPE | | BEST1 ≥ 6 (ddPCR) | | | ≥95% cobble | | Threshold | | | |
| cell lines exhibiting | | RPE65 ≥ 0.5 (ddPCR) | | | stone | | | | | |
| [symbol] | | | | | [symbol] | | | | | |
| Presence | | BEST1: 9.14 | | | NA | | 19F1i3R1 p3 | | passaging | AiPSC-RPE |
| | | RPE65: 0.309 FAIL | | | | | | | all cells | products |
| Presence | | BEST1: 10.9 | | | NA | | 19F1i4R1 p3 | | or 1 min | |
| | | RPE: 0.394 FAIL | | | | | | | or 3 min | |
| Presence | | NA | | | NA | | 19F1i10R3 p3 | | after | |
| | | NA | | | | | | | TrypLE | |
| Presence | | BEST1: 18.9 | | | pass | | 23F1i1R1b p3 | | | |
| | | RPE65: 0.357 FAIL | | | | | | | | |
| Presence | | BEST1: 17.9 | | | pass | | 23F1i1R2 p3 | | | |
| | | RPE65: 0.813 | | | | | | | | |
| Presence | | BEST1: 14.5 | | | pass | | 23F1i1R1 | | | |
| | | RPE65: 0.548 | | | | | 3 min p4 | | | |
| Presence | | BEST1: 10.2 | | | pass | | 23F1i3R1 p4 | | | |
| | | RPE65: 0.316 FAIL | | | | | | | | |
| Presence | | BEST1: 14.4 | | | pass | | 23F1i1R1 | | 10 min | |
| | | RPE65: 0.639 | | | | | 10 min p4 | | differential | |
| Presence | | BEST1: 28.8 | | | pass | | 23F1i3R2 p5 | | passaging | |
| | | RPE65: 1.63 | | | | | | | technique | |
| Presence | | BEST1: 18.3 | | | pass | | 23F1i1R5 p3 | | | |
| | | RPE65: 0.842 | | | | | | | | |
| | | Absence | Expression | Trypan | | STR | Human | | Sterility | |
| TEER | *% RPE | of [symbol] | of [symbol] | blue | Vector | (Short [symbol] | and [symbol] | Mycoplasma | per [symbol] | G-band |
| ≥150 | Pass: 80% | PASS: | ≥80% cells | ≥70% | PASS ≤ | PASS: No | Negative | No | No | Normal |
| ohms/cm2 | | LIN28 ≤ | express | live | 0.001% | contaminatio | | mycoplasma | microbial | karyotype |
| [symbol] | | 0.002% | [symbol] | cells | reprogrammi | [symbol] | | [symbol] | growth (or) | [symbol] |
| | | | | | [symbol] | | | | [symbol] | |
| 187 | 83.80% | PASS | PASS | 93.2% | N/A | | Negative | Negative | | pass: 18/20 |
| | | | | (NC200) | | | | | | |
| 31.02 FAIL | 92% | PASS | PASS | 81.2% | N/A | | Negative | Negative | | pass: 17/20 |
| | | | | (NC200) | | | | | | |
| 350 | 98% | ND | PASS | N/A | N/A | | Negative | Negative | | 18/20 |
| | | | | | | | | | | normal, one |
| | | | | | | | | | | [symbol] |
| 51.92 FAIL | 86.10% | PASS | PASS | 86.3% | N/A | | Negative | Negative | | pass: 19/20 |
| | | | | (NC200) | | | | | | |
| 110 FAIL | 92.50% | PASS | PASS | 83.0% | N/A | | Negative | Negative | | pass: 17/20 |
| | | | | (NC200) | | | | | | |
| 146 FAIL | 96.70% | PASS | PASS | 93.6% | N/A | | Negative | Negative | | pass 18/20 |
| | | | | (NC200) | | | | | | |
| 177.3 | 97.80% | PASS | PASS | 89% | N/A | | Negative | Negative | | pass: 14/20 * |
| | | | | (TC20) | | | | | | new |
| | | | | | | | | | | [symbol] |
| 228 | 94% | PASS | PASS | 94.7% | N/A | | Negative | Negative | | pass: 18/20 |
| | | | | (NC200) | | | | | | |
| 173.25 | 98.30% | PASS | PASS | 94.7% | N/A | | Negative | N/A | | pass: 14/20* |
| | | | | (NC200) | | | | | | new |
| | | | | | | | | | | [symbol] |
| | 98.00% | N/A | pass | 75% | N/A | | N/A | N//A | | pass |
| | | | | (TC20) | | | | | | |

| Phagocytosis- | | |
|---|---|---|
| [symbol] | Differential | |
| ≥70% of cells | Ratio | Ratio |
| phagocytose | of [symbol] | of [symbol] |
| 95.30% | 4.2 | 0.554 |
| | | [symbol] |
| 76.40% | 2.81 | 0.793 |
| | | [symbol] |
| 80.90% | 5.61 | 0.478 |
| | | [symbol] |
| 73.60% | 3.8 | 0.93 |
| | | FAIL |
| 84.20% | 2.85 | 1.16 |
| | | 72 hr |
| 89.30% | 3.2 | 1.06 |
| | | 72 hr |
| 99.10% | 2.19 | 1.09 |
| | | 72 hr |
| 94.40% | 3.04 | 1.08 |
| | | 72 hr |
| 97.30% | 4.23 | 1.01 |
| 99.50% | N/A | N/A |

[symbol]

indicates data missing or illegible when filed

Cells were then re-plated at a density of 10,000 cells/cm² for culture expansion and incubated overnight at 37 °C in a 5% CO₂. The propagation and purification of RPEs continued for approximately 12 weeks with cell morphology being checked at each passage, using a novel morphology scoring system developed to determine the optimal phenotype of AiPSC-RPE products. The scoring system helped guide key decisions in the manufacture process to produce quality RPE cells. AiPSC-RPE cells were grown in 6 well plates and five random images were taken to score the cells based on the morphology scoring system shown in FIG. 2. The scoring system was used to determine the number of passages to culture AiPSC-RPE to yield a final product that passed identity, safety, purity, potency and stability criteria. This system eliminated donor to donor variability and the results yielded an effective, potent and consistent product. Table 4 below, shows two AiPSC-RPE products at passage 3 that did not pass the morphology scoring system. However, when passaged to passage four, the cells not only passed the morphology scoring system but also passed all additional quality criteria.

TABLE 4
The Morphology Scaling System guides manufacturing to superior AiPSC-RPE products passing
all quality testing

| | | | AiPSC-RPE products do not pass | | AiPSC-RPE products do pass | |
|---|---|---|---|---|---|---|
| | | | morphology scoring system | | morphology scoring system | |
| | | | 23F1i1R1 | 23F1i1R1 | 23F1i1R1 | 23F1i1R1 |
| Purpose | Testing parameter | Threshold | 3 min p3 | 10 min p3 | 3 min p4 | 10 min p4 |
| Phenotype | Microscopy cell count | ≥95% cobble | did not pass | did not pass | pass | pass |
| | for cobble stone | stone | | | | |
| | morphology of RPE | morphology of | | | | |
| | cells | RPE cells | | | | |
| Gene | ddPCR for RPE | BEST1 ≥ 6 | BEST1: 18.5 | BEST1: 14.5 | BEST1: 14.5 | BEST1: 14.4 |
| expression | specific marker | (ddPCR) | | | | |
| | expression (RPE65; | | | | | |
| | BEST1; RLBP; | | | | | |
| | MerTK) | | | | | |
| | | RPE65 ≥ 0.5 | RPE65: 0.371 | RPE65: 0.548 | RPE65: 0.548 | RPE65: 0.639 |
| | | (ddPCR) | FAIL | | | |
| | | RLBP ≥ 1 | RLBP1: 2.5 | RLBP1: 2.6 | RLBP1: 2.6 | RLBP1: 2.89 |
| | | (ddPCR) | | | | |
| | | MerTK ≥ 0.15 | MerTK: 0.335 | MerTK: 0.297 | MerTK: 0.297 | MerTK: 0.294 |
| | | (ddPCR) | | | | |
| Protein | IHC for RPE markers | PASS: AiPSC- | Presence | Presence | Presence | Presence |
| Expression | (MITF, BEST1, ZO1) | RPE cell lines | | | | |
| | | exhibiting | | | | |
| | | expression | | | | |
| | | patterns equal | | | | |
| | | to positive | | | | |
| | | control | | | | |
| | | Lonza(primary | | | | |
| | | RPE cell line) | | | | |
| Safety | G-band karyotype | Normal | pass 18/20 | pass: 18/20 | pass: 18/20 | pass: 18/20 |
| | | karyotype | | | | |
| | | (PASS): ≥16 | | | | |
| | | normal cells/20 | | | | |
| | | total G-Banded | | | | |
| | Sterility per <USP71> | No microbial | NA | NA | NA | NA |
| | | growth (or) | | | | |
| | | Negative | | | | |
| | Mycoplasma by PCR | No mycoplasma | Negative | Negative | Negative | Negative |
| | | (or) Negative | | | | |
| | Human and murine | Negative | Negative | Negative | Negative | Negative |
| | viruses | | | | | |
| | STR (Short Tandem | PASS: No | PASS | PASS | PASS | PASS |
| | Repeats)—test for | contamination | | | | |
| | contamination | | | | | |
| | Vector Clearance by | PASS ≤ 0.001% | NA | NA | N/A | N/A |
| | ddPCR | reprogramming | | | | |
| | | vector copies in | | | | |
| | | RPE cells | | | | |
| | | analyzed by | | | | |
| | | ddPCR. | | | | |
| Stability: | Trypan blue cell count | PASS ≥ 70% | PASS | PASS | PASS | PASS |
| Viability to | | live cells | | | | |
| 6 hours | | | | | | |
| Purity | Expression of RPE | ≥80% cells | PASS | PASS | PASS | PASS |
| | marker PMEL17 by | express | | | | |
| | Imaging flow | PMEL17 | | | | |
| | cytometry | | | | | |
| | Absence of iPSC | PASS: LIN28 ≤ | PASS | PASS | PASS | PASS |
| | marker (Lin 28) by | 0.002% | | | | |
| | ddPCR | | | | | |
| | *% RPE (unique | Pass: 80% | N/D | N/D | 93.6% | 94.7% |
| | combination markers) | | | | (NC200) | (NC200) |
| | (PMEL17+CD140b+ | | | | | |
| | CD184−GD2−) | | | | | |
| Functionality | TEER measurements— | ≥150 | 99% | 99% | 96.70% | 94.00% |
| | Measuring tight | ohms/cm2 | | | | |
| | junctions for cell to cell | between 1 to 8 | | | | |
| | interactions | weeks in | | | | |
| | | culture. | | | | |
| | | | PASS | PASS | PASS | PASS |
| | Differential Secretion | Ratio of | 98.70% | 99.10% | 93.40% | 91.90% |
| | of PEDF/VEGF— | VEGF(Basal/ | | | | |
| | exhibit polarization of | Apical) ≥ 1 | | | | |
| | RPEs | | | | | |
| | | Ratio of PEDF | 96.58 FAIL | 134.64 FAIL | 146 FAIL | 228 |
| | | (Apical/ | | | | ohms/cm2 |
| | | Basal) ≥ 1 | | | | |
| | Phagocytosis—exhibit | ≥70% of cells | 92.10% | 96.80% | 89.30% | 94.40% |
| | functionality of RPE | phagocytosis of | | | | |
| | | rod segments | | | | |
| | | and measured | | | | |
| | | by flow | | | | |
| | | cytometry | | | | |
| | Differential Secretion | Ratio of | 0.838 FAIL | 0.774 FAIL | 1.06 72 hr | 1.08 72 hrs |
| | of PEDF/VEGF | VEGF(Basal/ | | | | |
| | | Apical) ≥ 1 | | | | |
| | | Ratio of PEDF | N/D | N/D | 3.2 | 3.04 |
| | | (Apical/ | | | | |
| | | Basal) ≥ 1 | | | | |

Single-cell suspensions of RPE cells were cryopreserved in ProFreeze CDM per the manufacturer's protocol. A sample of the cells prior to freezing was collected for in-process DS release testing as shown in Table 5.

TABLE 5
Summary Results from methods of AiPSC-RPE characterization

| | | | Result Summary |
|---|---|---|---|
| | | | (n = Donor cell line |
| | | | passed/n = Donor cell |
| Purpose | Testing parameter | Threshold | line tested) |
| Phenotype | Microscopy cell count for | ≥95% cobble stone | 12/12: Pass |
| | cobble stone morphology of | morphology of RPE cells | |
| | RPE cells | | |
| Gene | ddPCR for RPE specific marker | BEST1 ≥ 6 (ddPCR) | 14/14: Pass |
| expression | expression (RPE65; BEST1; | | |
| | RLBP; MerTK) | | |
| | | RPE65 ≥ 0.5 (ddPCR) | 10/14: Pass |
| | | RLBP ≥ 1 (ddPCR) | 14/14: Pass |
| | | MerTK ≥ 0.15 (ddPCR) | 14/14: Pass |
| Protein | IHC for RPE markers (MiTF, | PASS: AiPSC-RPE cell lines | 15/15: Pass |
| Expression | BEST1, ZO1) | exhibiting expression | |
| | | patterns equal to positive | |
| | | control Lonza(primary | |
| | | RPE cell line) | |
| Safety | G-band karyotype | Normal karyotype | 14/15: Pass |
| | | (PASS): ≥16 normal | |
| | | cells/20 total G-Banded | |
| | Sterility per <USP71> | No microbial growth | 15/15: Pass |
| | | (or) Negative | |
| | Mycoplasma by PCR | No mycoplasma | 15/15: Pass |
| | | (or) Negative | |
| | Human and murine viruses | Negative | 5/5: Pass |
| | STR (Short Tandem Repeats)- | PASS: No contamination | 15/15: Pass |
| | test for contamination | | |
| | Vector Clearance by ddPCR | PASS ≤ 0.001% | 13/13: Pass |
| | | reprogramming vector | |
| | | copies in RPE cells | |
| | | analyzed by ddPCR. | |
| Stability: | Trypan blue cell count | ≥70% live cells | 14/14: Pass |
| Viability | | | |
| to 6 hours | | | |
| Purity | Expression of RPE marker | ≥80% cells express | 15/15: Pass |
| | PMEL17 by Imaging flow | PMEL17 | |
| | cytometry | | |
| | Absence of iPSC marker | PASS: LIN28 ≤ 0.002% | 13/13: Pass |
| | (Lin28) by ddPCR | | |
| | *% RPE (unique combination | Pass: 80% | 7/7: Pass |
| | markers) | | |
| | (PMEL17 + CD140b + | | |
| | CD184 − GD2−) | | |
| Functionality | TEER measurements - | ≥150 ohms/cm2 | 10/14: Pass |
| | Measuring tight junctions for | between 1 to 8 weeks | |
| | cell to cell interactions | in culture. | |
| | Differential Secretion of | Ratio of | 10/14: Pass |
| | PEDF/VEGF exhibit | VEGF(Basal/Apical) ≥ 1 | |
| | polarization of RPEs | Ratio of PEDF | 14/14: Pass |
| | | (Apical/Basal) ≥ 1 | |
| | Phagocytosis- exhibit | ≥70% of cells | 13/14: Pass |
| | functionality of RPE | phagocytosis of rod | |
| | | segments and measured | |
| | | by flow cytometry | |
| | *This combination of markers | | 12/12: Pass |
| | was composed by our group | | |
| | and allows for identification of | | |
| | a population superior RPE | | |

### Stage 4-Culturing Cells for 4-6 Weeks Before Final Formulation Yielded Improved AiPSC-RPE Products

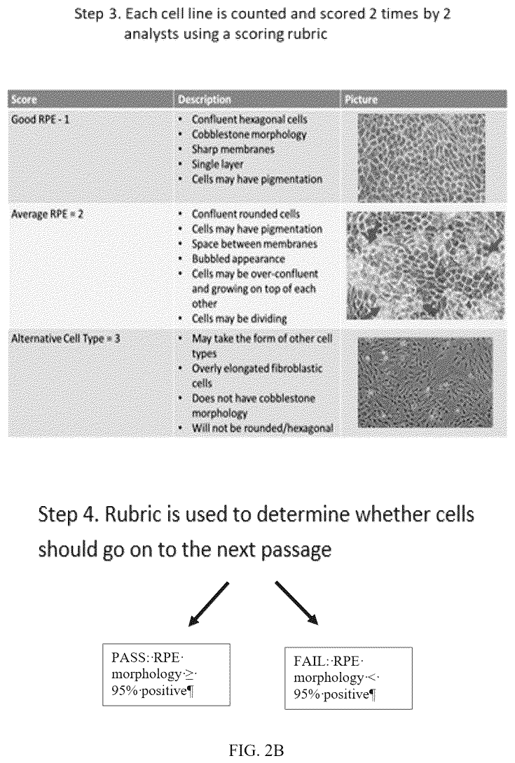

To understand optimal conditions for clinical formulation, freshly thawed AiPSC-RPE versus AiPSC-RPE cultured for four weeks were tested for viability and potency. All cells were re-suspended in balanced salt solution prior to testing, as intended for surgical delivery. AiPSC-RPE cultured for four weeks revealed a dramatic increase in both cell viability (FIG. 3A) and cell potency as measured by TEER (FIG. 3B) compared to freshly thawed AiPSC-RPE, leading to a significantly more favorable profile for transplantation, and were selected as the final formulation.

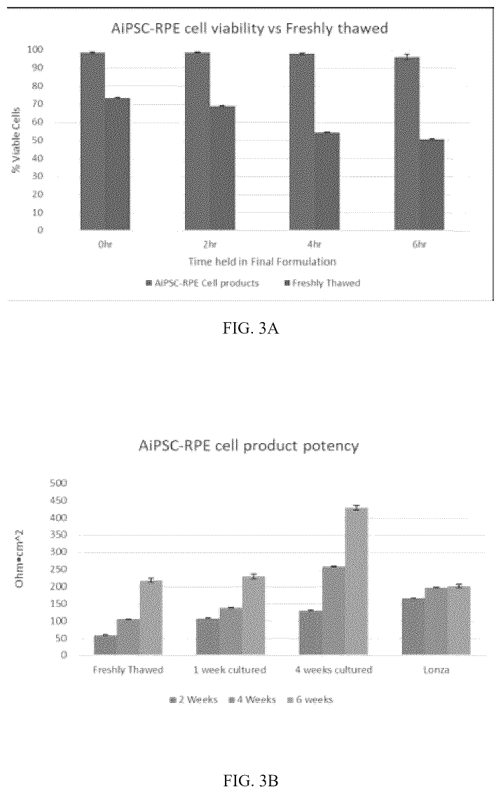

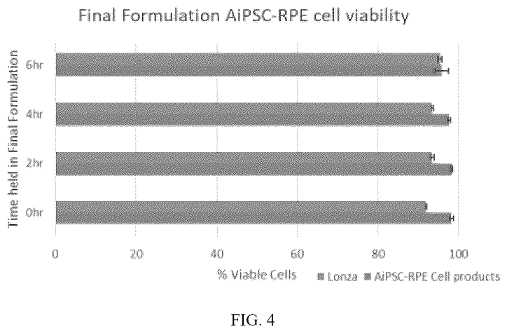

AiPSC-RPE drug products were tested in balanced salt solution formulation buffer, passing the cells through the delivery device and counting the cells for viability and potency at successive time periods. Cells had remarkable survivability post 6 hours after final formulation preparation, and although cell aggregation while in the salt solution was expected and observed, there was no impact to the number of cells recovered or the potency of the final product (Abe et al., Curr. Eye Res. 20 (4): 268-75 (2000); Lund et al., Cloning Stem Cells 8 (3): 189-99 (2006)). Additionally, viability by trypan blue dye exclusion and potency by differential secretion of cytokines VEGF and PEDF was constant, robust, and persisted up to 6 hours post passage through the delivery device. (See FIGS. 4 and 5; Lonza RPE was used as a primary cell line control). Together these data show a unique stability and potency of cell suspension products.

### Example 2-Identification of Unique RPE-Specific Protein Markers

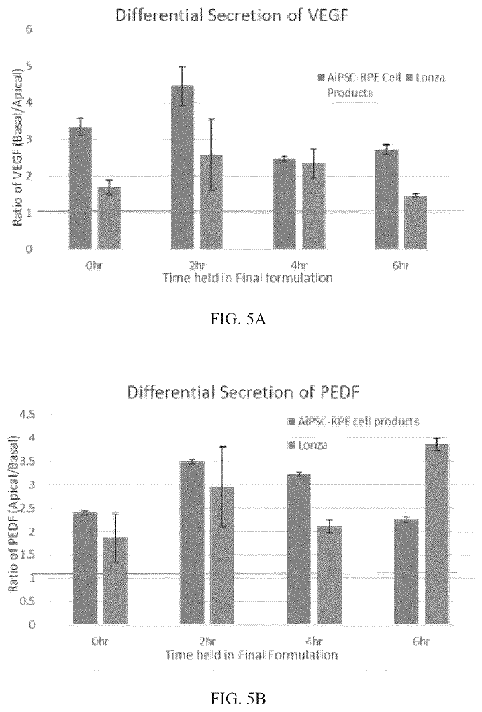

A unique panel of RPE specific markers was developed to show purity of AiPSC-RPE products. The protein expression profile included positivity to RPE markers: PMEL17, CD140b and negativity to non-RPE lineage markers, GD2 and CD184. As shown in FIGS. 6A-6D, purity of RPE cells, defined by increasing numbers of the PMEL17 (+)/CD140b (+)/GD2 (−)/CD184 (−) population, increased from passage 0 to passage 4 using TrypLE in Stage 3 differentiation process described in Example 1.

### Example 3-Evaluation of Identity, Safety and Potency of Stage 4 AiPSC-RPE Products

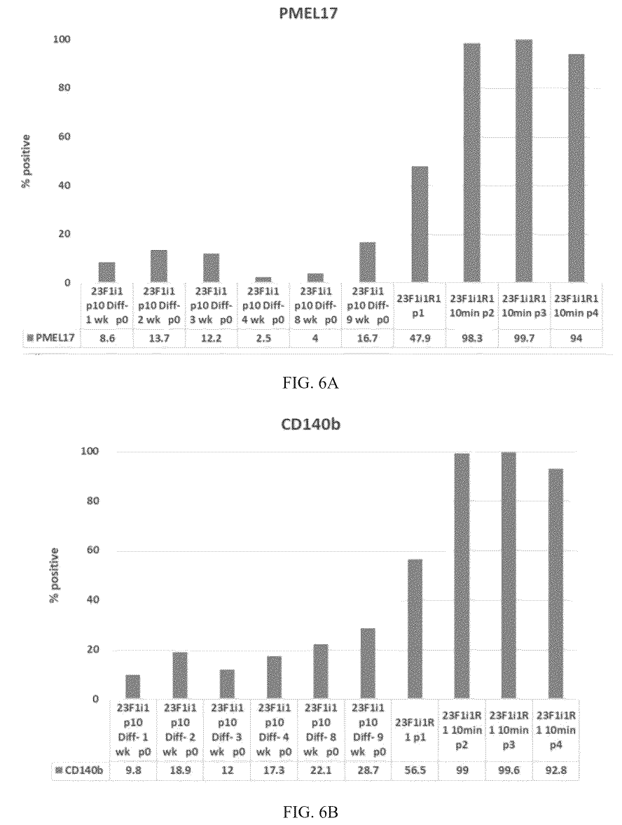

In order to determine variability and consistency between donor cultures, three patient specific AiPSC-RPE cell products produced from three parallel cultures of the same RPE bank, as shown in the schematic in FIG. 7 were tested for identity, safety and potency. To compare identity, gene expression was assessed using ddPCR. Additionally, a mycoplasma test was performed for safety evaluation, and a phagocytosis test on each parallel culture was used to assess potency.

The results presented in Table 6 show that parallel cultures were comparable in identity, safety, purity, and potency, and demonstrate that the parallel culture model is suitable for surrogate release testing of drug product (DP) to be administered to patients.

TABLE 6
Results of Comparability of Cultures from a Representative RPE Bank

| | Donor: | | | Donor: | | | |
|---|---|---|---|---|---|---|---|
| Donor: | 02F1i1R1 | | | 01F1i1R1 | | | |
| 06F1i1R1 | | | Gene | | | Gene | Plate |
| Phagocytosis | Phagocytosis | Mycoplasma | expression | Phagocytosis | Mycoplasma | expression | Test |
| Flow | Flow | | ddPCR gene | Flow | | ddPCR gene | |
| cytometry | cytometry | PCR | expression | cytometry | PCR | expression | Test Method |
| 270% of cells | ≥70% of cells | Negative | RPE65 ≥ 0.15 | ≥70% of cells | Negative | RPE65 ≥ | Threshold |
| phagocytose | phagocytose | | BEST1 ≥ 0.5 | phagocytose | | 0.15 | |
| | | | RLBP ≥ 0.5 | | | BEST1 ≥ | |
| | | | MerTK ≥ 0.15 | | | 6 | |
| | | | | | | RLBP ≥ | |
| | | | | | | [symbol] | |
| Potency | Potency | Safety | Phenotype | Potency | Safety | Phenotype | Purpose |
| 84.5% | 79.5% | Negative | RPE65: 0.697 | 86.7% | Negative | RPE65: | Parallel |
| | | | BEST1: 14.33 | | | 0.39 | Culture 1 |
| | | | RLBP: 0.77 | | | BEST1: | |
| | | | MerTK: 0.242 | | | 9.03 | |
| | | | | | | RLBP: | |
| | | | | | | [symbol] | |
| 87.4% | 79.3% | Negative | RPE65: 0.646 | 78.4% | Negative | RPE65: | Parallel |
| | | | BEST1: 14.33 | | | 0.43 | Culture 2 |
| | | | RLBP: 0.630 | | | BEST1: | |
| | | | MerTK: 0.241 | | | 9.29 | |
| | | | | | | RLBP: | |
| | | | | | | [symbol] | |
| 85.4% | 81.5% | Negative | RPE65: 0.607 | 81.4% | Negative | RPE65: | Parallel |
| | | | BEST1: 15.033 | | | 0.37 | Culture 3 |
| | | | RLBP: 0.643 | | | BEST1: | |
| | | | MerTK: 0.247 | | | 9.79 | |
| | | | | | | RLBP: | |
| | | | | | | [symbol] | |
| 1.73% | 1.52% | NA | 6.95% | 5.11% | NA | 7.90% | CVª (%) |
| | | | 1.45% | | | 4.18% | |
| | | | 18.72% | | | 3.65% | |
| | | | 1.07% | | | 3.04% | |

^{a}Coefficient of variation (CV) below 10% it is considered low, that is, the experiment has a high precision.
A coefficient of variation 10 to 20% is considered medium, implying the experiment has good precision. [symbol]
indicates data missing or illegible when filed

### Example 4-Evaluation of the Efficacy of Retinal Pigmented Epithelial Cells when Administered Sub-Retinally into the Royal College of Surgeons (RCS) Rat Model

To determine efficacy for AiPSC-RPE products in the RCS rat model, AiPSC-RPE products (manufactured as described in Example 1) from 3 independent patient donors were tested, as well one AiPSC-RPE product from a single patient donor (derived from separate iPSC lines from the single patient donor) in the RCS rat model of retinal degeneration. RCS rats are known for having genetic photoreceptor degeneration (Lund et al., Cloning Stem Cells 8 (3): 189-99 (2006)).

### Methods

Eighty-four RCS rats (48 males and 36 females) were assigned to the study. On postnatal day (P) 19-23, rats were assigned into 1 of 15 groups, per the study design in Table 7: Group 1, 6 and 12-AiPSC-RPE cells (01F1i1R1) at a dose of 60,000 cells/eye (treatment), Group 2, 7, and 13-AiPSC-RPE cells (02F1i1R1) at a dose of 60,000 cells/eye (treatment), Group 3, 8 and 14-AiPSC-RPE cells (13F1i1R1) at a dose of 60,000 cells/eye (treatment), Group 4, 9 and 15-AiPSC-RPE cells (13F1i2R1) at a dose of 60,000 cells/eye (treatment), Group 5-vehicle only (control), and Group 10-no treatment (control). Immunosuppressant treatment using dexamethasone (daily intraperitoneal injections for 14 days after dosing or assignment to study) and cyclosporine A (continuous administration via drinking water for remainder of study after dosing) was administered to all animals. Clinical ophthalmic examinations were performed at baseline and at Weeks 4, 8, and 12 after test article and vehicle control administration. OCT and color fundus photography were performed on Day 0 immediately post dose for animals in Groups 1 and 2 and at Weeks 4, 8, and 12 for all groups. The animals' visual acuity was tested with optokinetic response (OKR) and electroretinography (ERG) was performed in Weeks 4, 8, and 12. General health observations were performed and recorded daily. Body weights were measured at baseline, in Week 1, and prior to termination. Animals were terminated in Week 4, 8, or 12. Blood was collected for clinical pathology analysis, gross necropsies were performed on all animals, and eyes (whole globes), ocular adnexa, systemic tissues, and gross lesions were collected and analyzed.

TABLE 7
Efficacy Study Design to Test Persistence and Function of AiPSC-RPE Cell Products

| | | | | | | | | | Termination | |
|---|---|---|---|---|---|---|---|---|---|---|
| | | | | | | | Clinical | OCT & Color | Time Point | |
| | | Treatment | | Dose | | | Ophthalmic | Fundus | and Gross | Tissues |
| Group | N | OD | OS | (cells/eye) | OKR | ERG | Examinations | Photography | Necropsy | Collected |
| 1 | 6 | 01R1i1R1 | | 60,000 | Day | Day | Baseline & | Day 0 & Days | Day 56(±2) | Eyes OU |
| 2 | 6 | 02F1i1R1 | | | 26(±2) | 27(±2) | Days 28(±2) | 28(±2) | | (Whole |
| 3 | 6 | 13F1i1R1 | | | & Day | & Day | & 56(±2) | & 56(±2) | | Globes), |
| 4 | 6 | 13F1i2R1 | | | 54(±2) | 55(±2) | | | | Ocular |
| 5 | 6 | Vehicle | Untreated | N/A | | | | | | Adnexa. |
| 6 | 6 | 01R1i1R1 | | 60,000 | Day | Day | Baseline & | Day 0 & Days | Day 84(±2) | Selected |
| 7 | 6 | 02F1i1R1 | | | 26(±2), | 27(±2), | Days 28(±2), | 28(±2), | | Systemic |
| 8 | 6 | 13F1i1R1 | | | Day | Day | 56(±2), | 56(±2), | | Tissues, |
| 9 | 6 | 13F1i2R1 | | | 54(±2), | 55(±2), | & 84(±2) | & 84(±2) | | Whole |
| 10 | 6 | Vehicle | Untreated | N/A | & Day | & Day | | | | Blood & |
| | | | | | 82(±2) | 83(±2) | | | | Serum |

| | | | | | | | | |
|---|---|---|---|---|---|---|---|---|
| 12 | 6 | 01F1i1R1 | 60,000 | Day | Day | Baseline & | Day 0 & Days | Day 28(±2) |
| 13 | 6 | 02F1i1R1 | | 26(±2) | 27(±2) | Days 28(±2) | 28(±2) | |
| 14 | 6 | 13F1i1R1 | | | | | | |
| 15 | 6 | 13F1i2R1 | | | | | | |

### Results

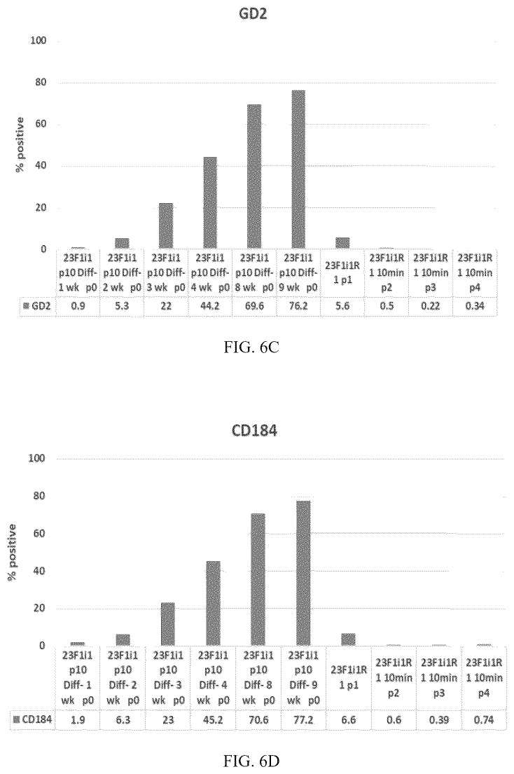

OCT was used to assess the thickness of the ONL at several time points. At four weeks post transplantation, there was a greater than 15% increase in ONL compared to control animals injected with vehicle alone across all AiPSC-RPE products tested. Also, at 8 weeks post transplantation, a greater than 5% increase in ONL was seen, compared to vehicle only controls across all AiPSC-RPE products (FIG. 8, adjusted p-values <0.01 for testing all patient-derived cell lines against vehicle alone, using Dunnett's method for multiple comparison). As the degradation of photoreceptors in the ONL of the RCS rat became increasingly advanced, no increase in ONL was seen, and results became inconclusive.

Additionally, human markers were used to examine the presence of transplanted cells in the sub retinal region. The persistence of AiPSC-RPE cells at all-time points measured, from week 4 through Week 12 post transplantation (Table 8), demonstrated robustness of the AiPSC-RPE cell products.

TABLE 8
Summary of Efficacy: Integration and persistence of transplanted
cells at 4, 8 and 12 weeks post transplantation across
four different AiPSC-RPE drug products

| | 4 Weeks Post | 8 Weeks Post | 12 Weeks Post |
|---|---|---|---|
| Sample | Injection ^{a} | Injection ^{a} | Injection ^{a} |
| AiPSC-RPE product 1 | 6/6 | 6/6 | 3/6 ^{b} |
| (01F1i1R1) | | | |
| AiPSC-RPE product 2 | 2/2 | 6/6 | 4/6 ^{b} |
| (02F1i1R1) | | | |
| AiPSC-RPE product 3 | 4/4 | 7/7 | 6/6 ^{b} |
| (13F1i1R1) | | | |
| AiPSC-RPE product 4 | 5/5 | 8/9 | 5/6 ^{b} |
| (13F1i2R1) | | | |
| Vehicle Control | 0/5 | 0/6 | 0/10 |
| Untreated | 0/2 | 0/6 | 0/12 |

^{a} Animals positive cells for human markers to identify AiPSC-RPE cell products are shown in the numerator.
^{b} Positive for migration and integration of AiPSC-RPE cell products

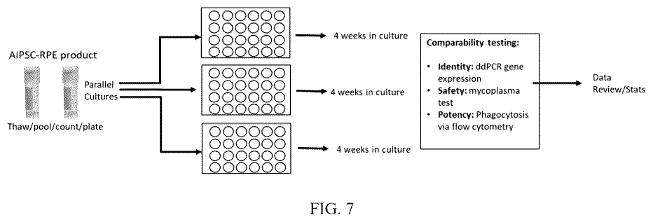

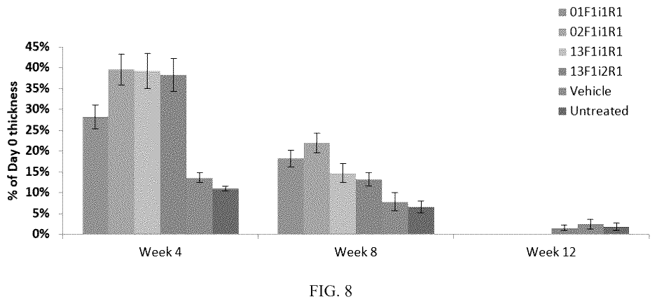

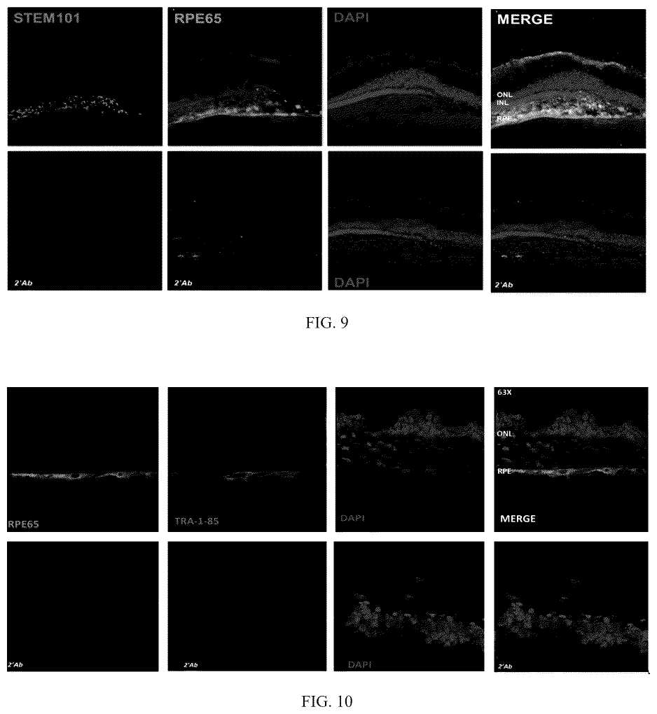

Immunocytochemistry showed protein expression of human nuclei at Week 4 and Week 12 post transplantation (FIG. 9 and FIG. 10, respectively). Week 8 results were consistent (data not shown). Importantly, at Week 12, evidence that AiPSC-RPE cells migrated from the area of injection (FIG. 11, white boxed area) and integrated in a mono layer with the endogenous RPE layer was seen. Cells also demonstrated polarization as evident by ezrin staining at the apical side (data not shown).

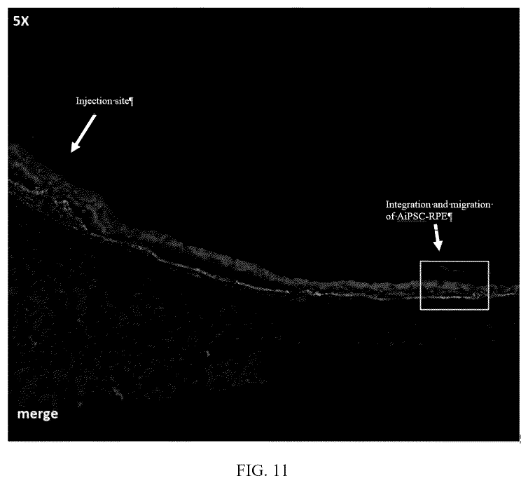

As a test for function in vivo, OKR was performed on the animal cohorts at time points up to 12 weeks post transplantation. OKR response in 01F1i1R1 had a greater than 7% increase at Week 8 and a 4% increase at Week 12. OKR response in 02F1i1R1 had a 3% increase for both Week 8 and 12 post transplantation. The highest increase in OKR response were seen in AiPSC-RPE cell products, 13F1i1R1 and 13F1i2R1, with a greater than 7% increase compared to the vehicle control only group throughout the study from Weeks 4-12 (FIG. 12, adjusted p-values <0.01 for testing all patient-derived cell lines against vehicle alone, using Dunnett's method for multiple comparison).

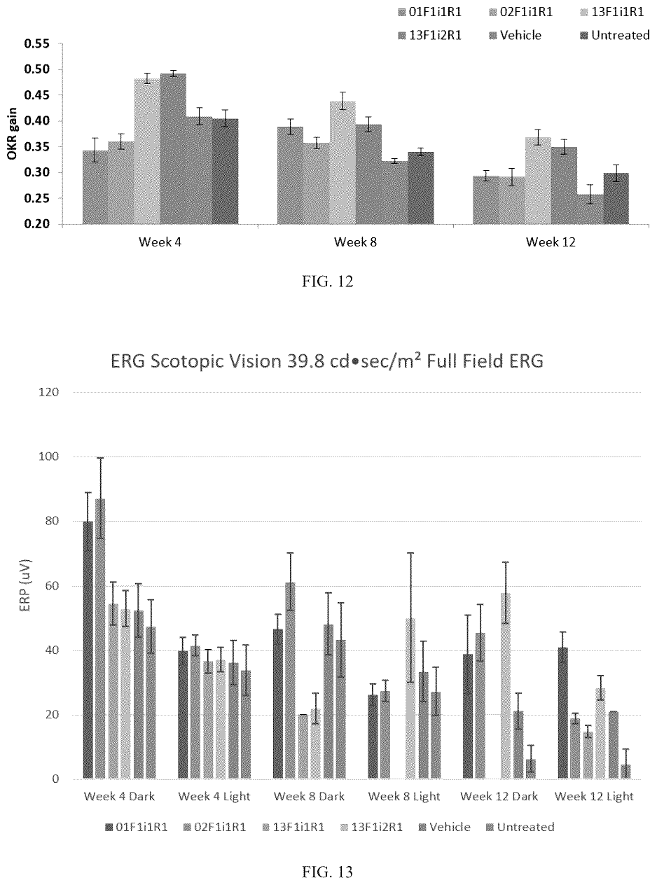

Additionally, ERG was used to test the function of the retina. In Week 4, scotopic b wave amplitude increased with increasing flash intensity for all treatment groups. At the highest intensity of 39.8 cd.see/m², scotopic b wave amplitude in eyes treated with 01F1i1R1 or 02F1i1R1 was higher compared to vehicle-treated and untreated eyes. Scotopic b wave amplitude at 39.8 cd.see/m² in eyes treated with 13F1i1R1 or 13F1i2R1 was comparable to vehicle-treated and uninjected eyes at this time point. By Week 8, scotopic b wave amplitude still increased with increasing flash intensity, but to a much smaller degree than in Week 4. At the highest intensity of 39.8 cd.see/m², scotopic b wave amplitude in eyes treated with 01F1i1R1 or 02F1i1R1 had decreased compared to Week 4. However, in eyes treated with 02F1i1R1, scotopic b wave amplitude was still slightly higher than in vehicle-treated and untreated eyes. The mean scotopic b wave amplitudes measured for eyes treated with 13F1i1R1 or 13F1i2R1 were lower than in other cell-injected eyes, but also slightly lower than in vehicle-treated and untreated eyes. By Week 12, scotopic b wave amplitude no longer increased consistently with increasing flash intensity. At the highest intensity of 39.8 cd.see/m², scotopic b wave amplitude in vehicle-treated and untreated eyes had decreased sharply compared to Week 8 and mean scotopic b wave amplitude in eyes treated with 01F1i1R1 and 13F1i2R1 was higher than in vehicle-treated and untreated eyes (FIG. 13).

### CONCLUSIONS

Sub-retinal injection of post-mitotic AiPSC-RPE cells from one of four different cell lines into the eyes of immunosuppressed RCS rats at a dose of 60,000 cells/eye was associated with formation of an AiPSC-RPE cell graft in the sub-retinal space and increased retinal ONL thickness at four and eight weeks after AiPSC-RPE cell implantation (at age ~P49 and ~P77, respectively) compared to vehicle-injected and untreated eyes. Pathological signs indicative of retinal degeneration, including retinal auto fluorescence, retinal vessel attenuation, RPE clumping, and pallor of the optic disc were also delayed and/or reduced in severity at these time points in eyes injected with AiPSC-RPE cells compared to vehicle-injected and untreated eyes. These findings suggest that treatment with AiPSC-RPE cells ameliorated photoreceptor degeneration and retinal deterioration characteristic of the RCS rat. Despite the RCS rats rapidly degrading retina we found positive functional results at multiple time points which indicate a potential therapeutic effect. Thus, the ERG analysis and assessment of OKR suggested possible retinal functional improvement in dystrophic RCS rats after RPE cell implantation, evidenced by enhanced ERG b waves and higher OKR gains.

Persistence and function of AiPSC-RPE cells were tested in the RCS rat model of retinal degeneration, a reasonably characterized animal model for dysfunction of RPEs in vivo. Both robust persistence and suitable function of the AiPSC-RPE DP was observed.

Multiple safety, toxicity and tumorigenicity studies were performed in SCID-beige mice and RNU rats (immune compromised animals) and found no adverse effects of the AiPSC-RPE products up to 3 months post transplantation in both the route of administration and in the testes, (a highly permissive tissue to measure tumors).

### OTHER EMBODIMENTS

While a number of embodiments of this invention are described herein, the present disclosure and examples may be altered to provide other methods and compositions of this invention. Therefore, it will be appreciated that the scope of this invention is to be defined by the appended claims in addition to the specific embodiments that have been represented by way of example. All references cited herein are hereby incorporated by reference.

1-37. (canceled)

38. A preparation of retinal pigment epithelium (RPE) cells differentiated from induced pluripotent stem cells (iPSCs), wherein:
(a) at least 80% of the RPE cells are PMEL17 (+)/CD140b (+)/GD2 (−)/CD184 (−) as determined by flow cytometry;
(b) the RPE cells express RPE65, BEST1, and RLBP1, wherein:
(i) gene expression of RPE65 is greater than or equal to 0.5, as assessed using droplet digital PCR (ddPCR),
(ii) gene expression of BEST1 is greater than or equal to 6, as assessed using ddPCR, and
(iii) gene expression of RLBP1 is greater than or equal to 1, as assessed using ddPCR; and
(c) less than 0.002% of RPE cells express LIN28.

39. The preparation of claim 38, wherein at least 80% of the RPE cells express MerTK.

40. The preparation of claim 38, wherein at least 85% of the RPE cells are PMEL17 (+)/CD140b (+)/GD2 (−)/CD184 (−).

41. The preparation of claim 40, wherein at least 85% of the RPE cells express MerTK.

42. The preparation of claim 38, wherein at least 90% of the RPE cells are PMEL17 (+)/CD140b (+)/GD2 (−)/CD184 (−).

43. The preparation of claim 42, wherein at least 90% of the RPE cells express MerTK.

44. The preparation of any claim 38, wherein a culture of the preparation comprises at least 95% of RPE cells exhibiting confluent hexagonal cells, pigmentation, cobblestone configuration, well-defined membrane borders, and polarization.

45. The preparation of claim 38, wherein:
(a) the RPE cells exhibit trans-epithelial electrical resistance (TEER) of at least 100 ohms/cm² after culturing for about 1 week to about 8 weeks;
(b) the RPE cells exhibit differential secretion of VEGF at a basal to apical secretion ratio greater than 0.5;
(c) the RPE cells exhibit differential secretion of PEDF at an apical to basal secretion ratio greater than 0.5; and
(d) at least 50% of the RPE cells exhibit ability to phagocytose photoreceptor outer segments.

46. The preparation of claim 38, wherein the iPSCs are derived from fibroblasts or from peripheral blood mononuclear cells.

47. A preparation of retinal pigment epithelium (RPE) cells differentiated from induced pluripotent stem cells (iPSCs), wherein:
(a) at least 80% of the RPE cells are PMEL17 (+)/CD140b (+)/GD2 (−)/CD184; and
(b) at least 80% of the RPE cells express RPE65, BEST1, and RLBP1.

48. The preparation of claim 47, wherein <0.002% of the RPE cells are positive for Lin28.

49. The preparation of claim 48, wherein at least 80% of the RPE cells express MerTK.

50. The preparation of claim 47, wherein:
(a) at least 85% of the RPE cells are PMEL17 (+)/CD140b (+)/GD2 (−)/CD184 (−); and
(b) at least 85% of the RPE cells express RPE65, BEST1, and RLBP1.

51. The preparation of claim 50, wherein <0.002% of the RPE cells are positive for Lin28.

52. The preparation of claim 51, wherein at least 85% of the RPE cells express MerTK.

53. The preparation of claim 47, wherein:
(a) at least 90% of the RPE cells are PMEL17 (+)/CD140b (+)/GD2 (−)/CD184 (−); and
(b) at least 90% of the RPE cells express RPE65, BEST1, and RLBP1.

54. The preparation of claim 53, wherein <0.002% of the RPE cells are positive for Lin28.

55. The preparation of claim 54, wherein at least 90% of the RPE cells express MerTK.

56. The preparation of any claim 47, wherein a culture of the preparation comprises at least 95% of RPE cells exhibiting confluent hexagonal cells, pigmentation, cobblestone configuration, well-defined membrane borders, and polarization.

57. The preparation of claim 47, wherein:
(a) the RPE cells exhibit trans-epithelial electrical resistance (TEER) of at least 100 ohms/cm² after culturing for about 1 week to about 8 weeks;
(b) the RPE cells exhibit differential secretion of VEGF at a basal to apical secretion ratio greater than 0.5;
(c) the RPE cells exhibit differential secretion of PEDF at an apical to basal secretion ratio greater than 0.5; and
(d) at least 50% of the RPE cells exhibit ability to phagocytose photoreceptor outer segments.

58. The preparation of claim 47, wherein the iPSCs are derived from fibroblasts or from peripheral blood mononuclear cells.

59. A method of manufacturing the preparation of claim 38, the method comprising:
propagating fibroblasts from a skin sample or propagating peripheral blood mononuclear cells from a whole blood sample;
producing iPSCs from the fibroblasts or from the peripheral blood mononuclear cells;
differentiating the iPSCs into a culture of RPE cells; and
passaging the culture of RPE cells for at least 10 minutes using a dissociation reagent until at least 95% of the cultured RPE cells exhibit confluent hexagonal cells, pigmentation, cobblestone configuration, and well defined membrane borders, thereby producing the preparation of RPE cells.

60. A method of manufacturing the preparation of claim 47, the method comprising:
propagating fibroblasts from a skin sample or propagating peripheral blood mononuclear cells from a whole blood sample;
producing iPSCs from the fibroblasts or from the peripheral blood mononuclear cells;
differentiating the iPSCs into a culture of RPE cells; and
passaging the culture of RPE cells for at least 10 minutes using a dissociation reagent until at least 95% of the cultured RPE cells exhibit confluent hexagonal cells, pigmentation, cobblestone configuration, and well defined membrane borders, thereby producing the preparation of RPE cells.

61. A method of treating a subject suffering from or at risk of an eye disorder, the method comprising administering to an eye of the subject the preparation of RPE cells of claim 38, thereby treating the subject.

62. The method of claim 61, wherein the RPE cells are autologous to the subject.

63. The method of claim 61, wherein the preparation comprises about 10,000 to about 1,500,000 RPE cells.

64. A method of treating a subject suffering from or at risk of an eye disorder, the method comprising administering to an eye of the subject the preparation of RPE cells of claim 47, thereby treating the subject.

65. The method of claim 64, wherein the RPE cells are autologous to the subject.

66. The method of claim 64, wherein the preparation comprises about 10,000 to about 1,500,000 RPE cells.
